(19) United States
(12) Patent Application Publication
(10) Pub. No.: US 2022/0305056 A1
(43) Pub. Date: Sep. 29, 2022

(54) CHIMERIC ANTIGEN RECEPTOR SYSTEM AND USES THEREOF

(71) Applicant: JANSSEN BIOTECH, INC., Horsham, PA (US)

(72) Inventors: Rajkumar GANESAN, Blue Bell, PA (US); Bharathikumar Vellalore MARUTHACHALAM, Downington, PA (US); Sathyadevi VENKATARAMANI, Blue Bell, PA (US); Iqbal GREWAL, Newtown, PA (US); Sanjaya SINGH, Blue Bell, PA (US); Paul HARVILLA, Nazareth, PA (US); Martin Jack BORROK III, Chalfont, PA (US); Ninkka TAMOT, Colmar, PA (US); Yonghai LI, Haddonfield, NJ (US)

(73) Assignee: JANSSEN BIOTECH, INC., Horsham, PA (US)

(21) Appl. No.: 17/637,946

(22) PCT Filed: Aug. 26, 2020
(86) PCT No.: PCT/US2020/047913

§ 371 Date: Feb. 24, 2022

Related U.S. Application Data
(60) Provisional application No. 62/705,780, filed on Jul. 15, 2020.
(60) Provisional application No. 62/892,225, filed on Aug. 27, 2019.

(51) Int. Cl. A61K 35/17; C07K 16/44; C07K 16/30; C07K 16/28; C07K 14/725; A61P 35/00

(52) U.S. Cl. CPC A61K 35/17; C07K 16/44; C07K 16/3069; C07K 16/28; C07K 14/7051; A61P 35/00; C07K 2317/31; A61K 39/00

(57) ABSTRACT

Provided herein are chimeric antigen receptors (CARs), cells comprising the CARs, monoclonal and bispecific antibodies or antigen-binding fragments thereof, and kits comprising the same. The CARs and bispecific antibodies are designed to target at least one tumor antigen and at least one tumor in methods of treating cancer in subjects in need thereof.

### CROSS REFERENCE TO RELATED APPLICATIONS

This application claims priority to U.S. Provisional Application No. 62/705,780, filed Jul. 15, 2020, and U.S. Provisional Application No. 62/892,225, filed Aug. 27, 2019. Each disclosure is incorporated herein by reference in its entirety.

### FIELD OF THE INVENTION

This disclosure relates to the molecular design strategies to overcome immune surveillance, heterogeneity, and antigen escape by tumor cells. More specifically, the disclosure relates to a modular CAR-T cell that anchors a binding moiety and a bispecific antibody to aid in tunable mono/multi-specificity for tumor targeting.

### REFERENCE TO SEQUENCE LISTING SUBMITTED ELECTRONICALLY

This application contains a sequence listing, which is submitted electronically via EFS-Web as an ASCII formatted sequence listing with a file name “065768.36WO1 Sequence Listing” and a creation date of Aug. 13, 2020 and having a size of 114 kb. The sequence listing submitted via EFS-Web is part of the specification and is herein incorporated by reference in its entirety.

### BACKGROUND OF THE INVENTION

Immunotherapy offers a new way to treat solid tumor and other cancers (June et al., Science 359:1361-5 (2018); Mirzaei et al., Front. Immunol. 8:1850 (2017)). Biologics including monoclonal antibodies, T-cell redirection bispecific antibodies, check point blockade, and most recently Chimeric antigen receptor T-cell (CAR-T) has greatly improved the treatment of tumors. Currently, two CAR-T therapies have been approved by the FDA with more in the clinic (Anderson and Mehta, Expert. Rev. Hematol. 12:551-61 (2019)). However, recent successes with CAR-T based therapies are not without their drawbacks (Minutolo et al., Front. Oncol. 9:176 (2019); Kloss et al., Nat. Biotechnol. 31:71-5 (2013); Brudno and Kochenderfer, Blood 127:3321-30 (2016); and Porter et al., Sci. Transl. Med. 7:303ra139 (2015)). CAR-Ts are generated by collecting blood from a patient, extracting T-cells, and expressing a chimeric antigen receptor, commonly with single chain fragment variables (scFv) that target a tumor associated antigen (TAA). This reprograms the T-cells of the patient to specifically target tumor cells and destroy them (Eshhar et al., Proc. Natl. Acad. Sci. USA 90:720-4 (1993)). As CAR-T cell therapy becomes the common line of therapy and an option for more patients, cost of goods, compliance of manufacturing process, and patient affordability becomes a topic of discussion. To this end, universal/allogenic CAR-T would alleviate the need to manufacture donor specific CAR-T cells (Yang et al., Curr. Opin. Hematol. 22:509-15 (2015); Eyquem et al., Nature 543:113-7 (2017)).

Further, current approved CAR-Ts only target a single TAA, which is not effective against tumors with heterogeneous TAA expression and does not circumvent antigen loss (Brentjens, R. J. et al. CD19-targeted T cells rapidly induce molecular remissions in adults with chemotherapy-refractory acute lymphoblastic leukemia. Sci Transl Med 5, 177ra138 (2013)). Various groups have found ways to target multiple TAA either by including two antigen recognition sites in the CAR or via a universal immune receptor (Minutolo, N. G., Hollander, E. E. & Powell, D. J., Jr. The Emergence of Universal Immune Receptor T Cell Therapy for Cancer. Front Oncol 9, 176 (2019); Cho, J. H., Collins, J. J. & Wong, W. W. Universal Chimeric Antigen Receptors for Multiplexed and Logical Control of T Cell Responses. Cell 173, 1426-1438 e1411 (2018); Zhao, J., Lin, Q., Song, Y. & Liu, D. Universal CARs, universal T cells, and universal CART cells. J Hematol Oncol 11, 132 (2018)). This decoupled approach allows targeting of multiple TAAs with one single source of universal T cells. To this end, a versatile CAR-adaptor pair was designed, which is independent of an engineered CAR component, and is capable of concurrently binding tumor cells and CAR-T cells. Thus, provided herein is a bispecific that can target a peptide linker in the CAR stalk of a T cell and that also targets a TAA (Coloma, M. J. & Morrison, S. L. Design and production of novel tetravalent bispecific antibodies. Nat Biotechnol 15, 159-163 (1997)). This system, referred to as a Conduit CAR-T demonstrates tumor specific cytotoxicity in a dose dependent manner.

### BRIEF SUMMARY OF THE INVENTION

In one general aspect, the invention relates to a chimeric antigen receptor (CAR) system that comprises (1) a CAR-T construct comprising a target polypeptide linker peptide linked to a non-antigen binding single chain variable fragment (scFv), wherein the target polypeptide linker peptide is in the CAR stalk, and (2) a bispecific antibody comprising (a) a first antigen-binding site that binds to the target polypeptide linker peptide and (b) a second antigen-binding site that binds to a tumor associated antigen (TAA) on a cancer cell, such that the CAR-T cell and cancer cell are bound by the bispecific antibody, which bridges the cancer cell and the CAR-T cell to kill the cancer cell.

In another general aspect, the invention relates to a CAR system that comprises (1) a CAR-T construct comprising a single chain variable fragment (scFv) comprising an antigen-binding site for a target polypeptide linker peptide, and (2) a bispecific antibody comprising (a) the target polypeptide linker peptide linked to a non-antigen binding scFv and (b) a second-antigen binding site that binds to a tumor associated antigen (TAA) on a cancer cell, such that the CAR-T cell and cancer cell are bound by the bispecific antibody, which bridges the cancer cell and CAR-T cell to kill the cancer cell.

Provided herein are isolated monoclonal antibodies or antigen-binding fragments thereof that specifically bind a (G₄S)ₙ polypeptide linker, wherein n is at least 2. The monoclonal antibodies or antigen-binding fragments thereof can comprise a heavy chain complementarity determining region 1 (HCDR1), HCDR2, HCDR3, a light chain complementarity determining region 1 (LCDR1), LCDR2, and LCDR3, having the polypeptide sequences of SEQ ID NOs:1, 2, 3, 4, 5, and 6, respectively. In certain embodiments, the monoclonal antibody or antigen-binding fragment thereof of comprises a heavy chain variable region having a polypeptide sequence at least 95% identical to SEQ ID NO:7, or a light chain variable region having a polypeptide sequence at least 95% identical to SEQ ID NO:8. In certain embodiments, the heavy chain variable region comprises the polypeptide sequence of SEQ ID NO:7, and the light chain variable region comprises the polypeptide sequence of SEQ ID NO:8.

In certain embodiments, the monoclonal antibody or antigen-binding fragment thereof is a single chain variable fragment (scFv). The scFv can, for example, comprise an amino acid sequence selected from SEQ ID NO:29 or SEQ ID NO:30.

Also provided herein are isolated bispecific antibodies or antigen-binding fragments thereof comprising a first polypeptide component and a second polypeptide component, wherein (a) the first polypeptide component comprises (i) a first antigen-binding domain that specifically binds a (G₄S)ₙ polypeptide linker, wherein n is at least 2, or (ii) a non-antigen binding single chain variable fragment (scFv) and a (G₄S)ₙ polypeptide linker, wherein n is at least 2; and (b) the second polypeptide component comprises a second antigen-binding domain that specifically binds a tumor associated antigen (TAA), preferably a human TAA.

In certain embodiments, the first antigen-binding domain comprises a heavy chain complementarity determining region 1 (HCDR1), a HCDR2, a HCDR3, a light chain complementarity determining region 1 (LCDR1), a LCDR2, and a LCDR3 having the polypeptide sequences of SEQ ID NOs:1, 2, 3, 4, 5, and 6, respectively; and the second antigen-binding domain comprises a heavy chain complementarity determining region 1 (HCDR1), a HCDR2, a HCDR3, a light chain complementarity determining region 1 (LCDR1), a LCDR2, and a LCDR3.

In certain embodiments, the second antigen-binding domain specifically binds prostate-specific membrane antigen (PSMA), preferably human PSMA, or transmembrane protein with EGF-like and two follistatin-like domains 2 (TMEFF2), preferably human TMEFF2. The second antigen-binding domain can, for example, comprise a heavy chain complementarity determining region 1 (HCDR1), a HCDR2, a HCDR3, a light chain complementarity determining region having the polypeptide sequences of (a) SEQ ID NOs:19, 20, 21, 22, 23, and 24, respectively, or (b) SEQ ID NOs:92, 93, 94, 95, 96, and 97, respectively.

In certain embodiments, the first antigen-binding domain comprises a first heavy chain variable region having a polypeptide sequence at least 95% identical to SEQ ID NO:7, and a first light chain variable region having a polypeptide sequence at least 95% identical to SEQ ID NO:8; and the second antigen-binding domain having a second heavy chain variable region comprising a polypeptide sequence at least 95% identical to SEQ ID NO:25 or SEQ ID NO:90, and a second light chain variable region having a polypeptide sequence at least 95% identical to SEQ ID NO:26 or SEQ ID NO:91. The first antigen-binding domain can, for example, comprise a first heavy chain variable region having the polypeptide sequence of SEQ ID NO:7, and a first light chain variable region having the polypeptide sequence of SEQ ID NO:8; and the second antigen-binding domain can, for example, comprise a second heavy chain variable region having the polypeptide sequence of SEQ ID NO:25 or SEQ ID NO:90, and the second light chain variable region having the polypeptide sequence of SEQ ID NO:26 or SEQ ID NO:91.

In certain embodiments, the isolated bispecific antibody or antigen-binding fragment thereof comprises the amino acid sequences selected from SEQ ID NO:35 and SEQ ID NO:28, SEQ ID NO:36 and SEQ ID NO:28, SEQ ID NO:37 and SEQ ID NO:27, SEQ ID NO:38 and SEQ ID NO:27, SEQ ID NO: 101 and SEQ ID NO: 28, SEQ ID NO: 102 and SEQ ID NO: 28, SEQ ID NO: 103 and SEQ ID NO: 98, or SEQ ID NO: 104 and SEQ ID NO: 98.

In certain embodiments, the non-antigen binding scFv comprises a heavy chain complementarity determining region 1 (HCDR1), a HCDR2, a HCDR3, a light chain complementarity determining region 1, a LCDR2, and a LCDR3 having the polypeptide sequences of SEQ ID NOs:11, 12, 13, 14, 15, and 16, respectively. In certain embodiments, the non-antigen binding scFv comprises a heavy chain variable region having an amino acid sequence at least 95% identical to SEQ ID NO:17, and a light chain variable region having an amino acid sequence at least 95% identical to SEQ ID NO:18. The non-antigen binding scFv can, for example, comprise a heavy chain variable region having the amino acid sequence of SEQ ID NO:17, and a light chain variable region having the amino acid sequence of SEQ ID NO:18.

In certain embodiments, the (G₄S)ₙ linker peptide comprises an amino acid sequence selected from the group consisting of SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, and SEQ ID NO:55.

In certain embodiments, the monoclonal or bispecific antibody or antigen-binding fragment thereof is chimeric and/or human or humanized.

Also provided are isolated nucleic acids encoding the monoclonal or bispecific antibodies or antigen-binding fragments thereof as disclosed herein.

Also provided are isolated polynucleotides comprising a nucleic acid encoding a chimeric antigen receptor (CAR). The CAR can, for example, comprise (a) an extracellular domain comprising (1) a non-antigen binding single chain variable fragment (scFv) and a (G₄S)ₙ polypeptide linker or (2) an antigen binding domain that specifically binds a (G₄S)ₙ polypeptide linker; (b) a transmembrane region; and (c) an intracellular signaling domain.

In certain embodiments, the non-antigen binding scFv comprises a heavy chain complementarity determining region 1 (HCDR1), a HCDR2, a HCDR3, a light chain complementarity determining region 1, a LCDR2, and a LCDR3 having the polypeptide sequences of SEQ ID NOs:11, 12, 13, 14, 15, and 16, respectively. In certain embodiments, the non-antigen binding scFv comprises a heavy chain variable region having an amino acid sequence at least 95% identical to SEQ ID NO:17, and a light chain variable region having an amino acid sequence at least 95% identical to SEQ ID NO:18. The non-antigen binding scFv can, for example, comprise a heavy chain variable region having the amino acid sequence of SEQ ID NO:17, and a light chain variable region having the amino acid sequence of SEQ ID NO:18. The non-antigen binding scFv can, for example, comprise an amino acid sequence selected from SEQ ID NO:33 or SEQ ID NO:34.

In certain embodiments, the (G₄S)ₙ linker peptide comprises an amino acid sequence selected from the group consisting of SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, and SEQ ID NO:55.

In certain embodiments, the extracellular domain is a CD8 extracellular domain. The CD8 extracellular domain can, for example, comprise the amino acid sequence of SEQ ID NO:41. In certain embodiments, the transmembrane domain is a CD8 transmembrane domain. The CD8 transmembrane domain can, for example, comprise the amino acid sequence of SEQ ID NO:42. In certain embodiments, the intracellular signaling domain comprises a CD137 costimulatory domain and CD3ζ activating domain. The CD137 costimulatory domain can, for example, comprise the amino acid sequence of SEQ ID NO:43, and the CD3ζ activating domain can comprise the amino acid sequence of SEQ ID NO:44.

In certain embodiments, the CAR can comprise an amino acid sequence selected from SEQ ID NO:39 or SEQ ID NO:40.

In certain embodiments, the antigen-binding domain can comprise a heavy chain complementarity determining region 1 (HCDR1), HCDR2, HCDR3, a light chain complementarity determining region 1 (LCDR1), LCDR2, and LCDR3, having the polypeptide sequences of SEQ ID NOs:1, 2, 3, 4, 5, and 6, respectively. In certain embodiments, the antigen-binding domain can comprise a heavy chain variable region having a polypeptide sequence at least 95% identical to SEQ ID NO:7, or a light chain variable region having a polypeptide sequence at least 95% identical to SEQ ID NO:8. The heavy chain variable region can, for example, comprise the polypeptide sequence of SEQ ID NO:7, and the light chain variable region can, for example, comprise the polypeptide sequence of SEQ ID NO: 8.

In certain embodiments, the antigen-binding domain is a single chain variable fragment (scFv). The scFv can, for example, comprise an amino acid sequence selected from SEQ ID NO:29 or SEQ ID NO:30.

Also provided are chimeric antigen receptors (CARs) encoded by the isolated polynucleotides as disclosed herein.

Also provided are isolated vectors comprising the isolated nucleic acids or isolated polynucleotides as disclosed herein.

Also provided are isolated host cells comprising the isolated vectors as disclosed herein. The isolated host cells can, for example, comprise a T cell or a NK cell, preferably a human T cell or a human NK cell.

Also provided are methods of producing a chimeric antigen-receptor (CAR)-T cell or a CAR-NK cell. The methods can, for example, comprise culturing T cells or NK cells comprising the isolated polynucleotides encoding CARs as disclosed herein under conditions to produce a CAR-T cell or CAR-NK cell and recovering the CAR-T cell or CAR-NK cell.

Also provided are kits comprising (a) isolated polynucleotides comprising a nucleic acid encoding a chimeric antigen receptor (CAR) as disclosed herein, and (b) an isolated bispecific antibody or antigen-binding fragment thereof as disclosed herein.

Also provided are methods of treating a cancer expressing a tumor associated antigen (TAA) in a subject in need thereof. The methods comprise administering to the subject a CAR-T or CAR-NK cell as disclosed herein and a pharmaceutical composition comprising a bispecific antibody or antigen-binding fragment thereof as disclosed herein and a pharmaceutically acceptable carrier.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows a schematic of the mechanism of action of the conduit CAR-T system. T cells transfected with the universal CAR stalk contain an inert scFv with a G₄S linker on the cell surface. Tumor cells contain tumor specific antigens on the cell surface. Using a bridging bispecific antibody adaptor, the universal CAR-T cell homes in on the tumor cell by binding both the bridging bispecific antibody and the tumor specific antigen. By changing the tumor antigen targeting portion of the bridging bispecific antibody, it is possible to treat cancers and relapses using different tumor antigens while still using the same universal CAR-T cells.

FIG. 2 shows a graph providing the results of an enzyme linked immunosorbent assay (ELISA) showing binding of the CEN-63 C13 anti-G₄S antibody to immobilized scFv containing a G₄S linker (circles) and a non-G₄S linker (squares). The CEN-63 C13 anti-G₄S antibody demonstrated dose dependent binding and an EC₅₀ of 0.57 nM.

FIG. 3 shows a graph demonstrating CEN-63 C13 binding to cell-surface scFv with a G₄S linker but not to an scFv with a non-G₄S linker. Binding of CEN-63 C13 antibody to HEK293T cells transfected with CAR-T constructs harboring desired scFv domains was assessed using a fluorescently labelled anti-human Fc antibody. CEN-63-13 bound cell surface antigen in a dose dependent manner with a calculated EC₅₀ of 0.78 nM.

FIG. 4 shows a graph providing K_{D} values for CEN-63 C13 antibody binding to the WT (G₄S)₄ peptide linker and linker truncation variants. The minimal linker-1 length required for CEN-63 C13 binding was determined to be 10 amino acids. No binding was observed for linkers with less than 10 amino acids. Biotinylated-peptide linkers were immobilized onto streptavidin biosensors and binding of CEN-63 C13 to full-length and truncated linkers was measured using bio-layer interferometry.

FIG. 5 shows a schematic showing the design of bispecific antibody adaptor used in the conduit CAR-T platform. The table shows the tumor-binding and linker-binding arms of four bispecific antibody adaptors used for validating the conduit CAR-T platform.

FIGS. 6A-6D show the verification of conduit CAR-T expression. Conduit (G₄S)₄ CAR-T cells were made by electroporating activated primary human T lymphocytes with in vitro transcribed mRNA coding for the desired CAR-T construct. FIG. 6A shows the detection of conduit-CAR expression by staining transfected T cells with or without CEN-63 C13 antibody. FIG. 6B shows the comparison of conduit-CAR expression with or without bispecific antibody adaptors. The addition of adaptors did not affect conduit CAR expression. FIG. 6C shows isotype CAR-transgene expression was detected by anti-G₄S CEN-63-13 antibody followed by anti-human PE secondary antibody. Staining was performed 2 days following mRNA transduction of CD3+ Pan-T cells. FIG. 6D shows anti-CD19 CAR with an (G₄S)₃ linker containing an N-terminal MYC tag could be detected on lentiviral transfected Pan-T cells. MYC positive CAR-T cells had increased CEN-63-13 staining whereas MYC negative cells had little CEN-63-13 staining.

FIGS. 7A-7D show flow cytometry histograms showing the amount of CD69 activation in CD8+ T cells expressing the universal CAR stalk. FIG. 7A demonstrates that effector T cells alone in the presence of no target tumor cells showed baseline expression of CD69. FIG. 7B demonstrates that tumor cells added to Effector T cells increased expression of CD69 relative to effector T cells only. FIG. 7C demonstrates the maximum level of CD69 induction on Effector T cells occurred in the presence of both tumor cells and Conduit bispecific molecules. FIG. 7D shows a graph illustrating that highest levels of T cell activation occurred when universal CAR T cells were co-cultured with tumor cells in the presence of bispecific antibodies.

FIGS. 8A-8B show the validation of the conduit CAR-T platform. Conduit CAR cells killed tumor cells in the presence of bispecific antibody adaptors. FIG. 8A shows the analysis of CD107a expression upon incubating CAR-T cells with tumor cells for 4 hours. CD107a expression was measured by gating on CD8+ CAR+ and CD8+ CAR− cells. Bispecific molecules significantly activated CD107a expression. FIG. 8B shows cytolytic potential of bispecific molecules at different E:T ratios as measured by xCELLigence cytotoxicity assay. At a final concentration of 5 μM, all bispecific molecules showed potent cytotoxicity at higher E:T ratios.

FIGS. 9A-9C show bispecific cell binding, proliferation and ligand-engagement dependent proliferation & degranulation. FIG. 9A: the presence of BsAb alone does not alter CAR surface expression. BsAb molecules (5 μg/ml) were added into Isotype CAR-T cells with a (G₄S)₄ linker and incubated for 2 days. CAR-surface expression in CD3/CD4/CD8 positive T cells was detected by anti-G₄S antibody (CAR in CD8⁺ Tcells shown here) and remained similar in the presence or absence of bispecific antibodies. FIG. 9B: CAR-T cells generated using PanT cells from two different donors were labeled with CFSE, co-culture with PSMA expressing tumor cells in presence/absence of BsAb. CFSE staining intensity was analyzed 72 hours after stimulation. CFSE staining in absence of BsAb is shown in gray histograms and those in presence of BsAb in green histogram. FIG. 9C: (G₄S)₄-containing CAR-T cells were co-cultured with PSMA-expressed tumor cells with and without BsAb1. After 5 hours co-culture, CD107a detection of CAR-T cells was measured. CEN-63-13 mAb was used to detect CAR expression. Representative plots show CAR-negative cells had no CD107a expression in the presence or absence of BsAb1. In the CAR+ population, only cells incubated with bispecific antibody showed appreciably increased CD107a expression, suggesting BsAbs are necessary for CD107a expression in the presence of tumor cells.

FIGS. 10A-10D show dynamic monitoring of CAR-T-mediated cytotoxicity and cytokine profile. xCelligence cytotoxicity assay was used to measure real-time tumor cell lysis by CAR-T cell in presence of serial diluted BsAb. Bispecific anti-PSMA & anti-G₄S linker molecules (BsAb1 & 2) were generated, and further tested in xCelligence cytotoxicity assay targeting PSMA expressing PC3 cells (FIG. 10A). In this 97 hour experiment the E:T ratio of Isotype G₄S-containing CAR-T cells to PC3 cells was 5:1. Experiments were performed in triplicate. FIG. 10B: Percent cytoloysis at 72 hours at different E:T ratios were also accessed and are shown for BsAb1 & 2. FIG. 10C: Similar xCelligence cytotoxicity experiments were performed using anti-TMEFF2 and anti-G₄S linker bispecific antibodies. Dose dependent cytotoxicity was observed only in the presence of BsAb. FIG. 10D: Cytokine profile of supernatant in co-culture of CAR-T and tumor cells (E:T=5:1). INFγ, GMCSF and IL-6 levels were assessed at 72 hours after incubation and were elevated in the presence of bispecific antibodies. Data are shown as the mean±SD. Significance between groups containing both CAR-T and PC3 cells were calculated using one-way ANOVA with multiple comparisons (Tukey test), *p<0.05, ** p<0.01,*** p<0.001, ****p<0.0001.

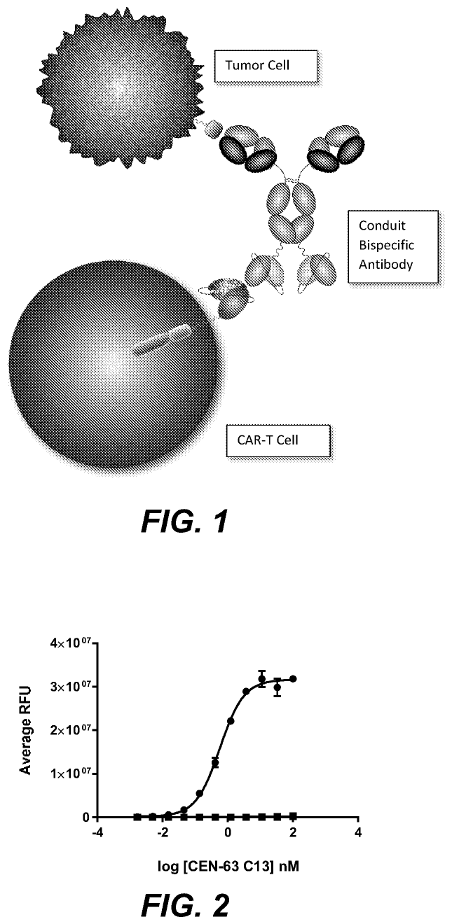

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of” or variations such as “consist of” or “consisting of” as used throughout the specification and claims, indicate the inclusion of any recited integer or group of integers, but that no additional integer or group of integers can be added to the specified method, structure, or composition.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., chimeric antigen receptors (CARs) and the isolated polynucleotides that encode them; isolated monoclonal or bispecific antibodies and antigen-binding fragments thereof and the nucleic acids that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected or transduced with a nucleic acid molecule of the invention. In another embodiment, a “host cell” is a progeny or potential progeny of such a transfected or transduced cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed CAR can be within the cytoplasm of a host cell, into the extracellular milieu such as the growth medium of a cell culture or anchored to the cell membrane.

As used herein, the term “immune cell” or “immune effector cell” refers to a cell that is involved in an immune response, e.g., in the promotion of an immune effector response. Examples of immune cells include T cells, B cells, natural killer (NK) cells, mast cells, and myeloid-derived phagocytes. According to particular embodiments, the engineered immune cells are T cells, and are referred to as CAR-T cells because they are engineered to express CARs of the invention.

As used herein, the term “engineered immune cell” refers to an immune cell, also referred to as an immune effector cell, that has been genetically modified by the addition of extra genetic material in the form of DNA or RNA to the total genetic material of the cell. According to embodiments herein, the engineered immune cells have been genetically modified to express a CAR construct according to the invention.

### Chimeric Antigen Receptor (CAR)

As used herein, the term “chimeric antigen receptor” (CAR) refers to a polypeptide comprising at least an extracellular domain that is bound by a monospecific or multispecific antibody or binds specifically to a target on a monospecific or multispecific antibody, a transmembrane domain and an intracellular T cell receptor-activating signaling domain. The extracellular domain can comprise a binding domain against a linker polypeptide, a linker polypeptide alone, or a linker polypeptide fused to a recombinant polypeptide. Engagement of the extracellular domain of the CAR with a multispecific antibody, which is engineered to bind a tumor associated antigen (TAA) on a cancer cell, results in clustering of the CAR and delivers an activation stimulus to the CAR-containing cell. CARs redirect the specificity of immune effector cells and trigger proliferation, cytokine production, phagocytosis and/or production of molecules that can mediate cell death of the TAA-expressing cell in a major histocompatibility (MHC)-independent manner.

In one aspect, the CAR comprises an extracellular domain comprising a non-antigen binding single chain variable fragment (scFv) and a (G₄S)ₙ polypeptide linker, wherein n is at least 2; a transmembrane region; and an intracellular signaling domain. In another aspect, the CAR comprises an extracellular domain comprising an antigen binding domain that specifically binds a (G₄S)ₙ polypeptide linker, wherein n is at least 2; a transmembrane region; and an intracellular signaling domain.

According to a particular aspect, the non-antigen binding scFv comprises a heavy chain complementarity determining region 1 (HCDR1), a HCDR2, a HCDR3, a light chain complementarity determining region 1, a LCDR2, and a LCDR3 having the polypeptide sequences of SEQ ID NOs:11, 12, 13, 14, 15, and 16, respectively.

According to another particular aspect, the non-antigen binding scFv can comprise a heavy chain variable region having an amino acid sequence at least 95%, at least 96%, at least 97%, at least 98%, at least 99%, or 100% identical to SEQ ID NO:17, and a light chain variable region having an amino acid sequence at least 95%, at least 96%, at least 97%, at least 98%, at least 99%, or 100% identical to SEQ ID NO:18.

According to another particular aspect, the non-antigen binding scFv can, for example, comprise an amino acid sequence selected from SEQ ID NO:33 or SEQ ID NO:34.

According to a particular aspect, the antigen-binding domain can comprise a heavy chain complementarity determining region 1 (HCDR1), HCDR2, HCDR3, a light chain complementarity determining region 1 (LCDR1), LCDR2, and LCDR3, having the polypeptide sequences of SEQ ID NOs:1, 2, 3, 4, 5, and 6, respectively.

According to another particular aspect, the antigen-binding domain can comprise a heavy chain variable region having a polypeptide sequence at least 95%, at least 96%, at least 97%, at least 98%, at least 99%, or 100% identical to SEQ ID NO:7, or a light chain variable region having a polypeptide sequence at least 95%, at least 96%, at least 97%, at least 98%, at least 99%, or 100% identical to SEQ ID NO:8.

According to another particular aspect, the antigen-binding domain is a single chain variable fragment (scFv). The scFv can, for example, comprise an amino acid sequence selected from SEQ ID NO:29 or SEQ ID NO:30.

According to another particular aspect, the extracellular domain can comprise a CD8 extracellular domain linked to (1) the non-antigen binding single chain variable fragment (scFv) and (G₄S)ₙ polypeptide linker or (2) the antigen binding domain that specifically binds the (G₄S)ₙ polypeptide linker. The CD8 extracellular domain can, for example, comprise the amino acid sequence of SEQ ID NO:41.

According to another particular aspect, the transmembrane domain is a CD8 transmembrane domain. The CD8 transmembrane domain can, for example, comprise the amino acid sequence of SEQ ID NO:42.

According to another particular aspect, the intracellular signaling domain comprises a CD137 costimulatory domain and a CD3ζ activating domain. The CD137 costimulatory domain can, for example, comprise the amino acid sequence of SEQ ID NO:43. The CD3ζ activating domain can, for example, comprise the amino acid sequence of SEQ ID NO:44.

According to another particular aspect, the CAR can comprise an amino acid sequence selected from SEQ ID NO:39 or SEQ ID NO:40.

According to another particular aspect, also provided herein are chimeric antigen receptors (CARs) encoded by the isolated polynucleotides as disclosed herein.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen binding domain,” “extracellular domain,” or “extracellular ligand binding domain” refers to the part of a CAR that is located outside of the cell membrane and is capable of binding to an antigen, target, or ligand, or, alternatively, is capable of being bound by an antigen-binding domain that specifically recognizes a portion of the extracellular domain (e.g., a polypeptide linker that is capable of being specifically bound by an antibody or antigen-binding fragment thereof).

As used herein, the term “hinge region” refers to the part of a CAR that connects two adjacent domains of the CAR protein, e.g., the extracellular domain and the transmembrane domain.

As used herein, the term “transmembrane domain” refers to the portion of a CAR that extends across the cell membrane and anchors the CAR to cell membrane.

As used herein the term “intracellular signaling domain” refers to the portion of a CAR that is inside the cell membrane that acts to activate the signaling cascade when the extracellular domain of the CAR is engaged. The intracellular signaling domain can, for example, comprise a costimulatory domain and an activating domain.

### Costimulatory Domains

As used herein, chimeric antigen receptors can incorporate costimulatory (signaling) domains to increase their potency. A costimulatory (signaling) domain can be derived from a costimulatory molecule. Costimulatory molecules are cell surface molecules other than antigen receptors or their ligands that are required for an efficient immune response. Costimulatory domains can be derived from costimulatory molecules, which can include, but are not limited to CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD33, CD37, CD40, CD45, CD64, CD80, CD86, CD134, CD137, CD154, programmed death-1 (PD-1), inducible T cell costimulator (ICOS), lymphocyte function-associated antigen-1 (LFA-1; CD11a and CD18), CD247, CD276 (B7-H3), LIGHT (tumor necrosis factor superfamily member 14; TNFSF14), NKG2C, Ig alpha (CD79a), DAP10, Fc gamma receptor, MHC class I molecule, TNFR, integrin, signaling lymphocytic activation molecule, BTLA, Toll ligand receptor, ICAM-1, CD5, GITR, BAFFR, LIGHT, HVEM (LIGHTR), KIRDS2, SLAMF7, NKp80 (KLRF1), NKp44, NKp30, NKp46, CD19, CD8 alpha, CD8 beta, IL-2R beta, IL-2R gamma, IL-7R alpha, ITGA4, VLA1, CD49a, IA4, CD49D, ITGA6, VLA-6, CD49f, ITGAD, ITGAE, CD103, ITGAL, CD1a, CD1b, CD1c, CD1d, ITGAM, ITGAX, ITGB1, CD29, ITGB2 (CD18), ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, cytokine receptor, activating NK cell receptors, or fragments or any combination thereof. In a preferred embodiment, the costimulatory domain is a CD137 costimulatory domain.

### Activating Domains

As used herein, chimeric antigen receptors can comprise activating domains. Activating domains can include, but are not limited to, CD3. CD3 is an element of the T cell receptor on native T cells and has been shown to be an important intracellular activating element in CARs. In a preferred embodiment, the CD3 is CD3 zeta (c).

### Hinge Region

As described herein, the chimeric antigen receptor can comprise a hinge region. This is a portion of the extracellular domain, sometimes referred to as a “spacer” region. A variety of hinges can be employed in accordance with the invention, including costimulatory molecules, as discussed above, immunoglobulin (Ig) sequences, or other suitable molecules to achieve the desired special distance from the target cell. In some embodiments, the entire extracellular region comprises a hinge region.

In some embodiments, the extracellular domain comprises a hinge region, wherein the hinge region is a polypeptide linker sequence. In certain embodiments, the hinge region comprises a (G₄S)ₙ linker peptide. In certain embodiments, the (G₄S)ₙ linker peptide can be operably linked to a non-antigen binding scFv.

The (G₄S)ₙ linker peptide can, for example, comprise an amino acid sequence selected from the group consisting of SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, and SEQ ID NO:55. In a preferred embodiment, the (G₄S)ₙ linker peptide comprises the amino acid sequence of SEQ ID NO:45. Examples of polypeptide linker sequences can be found in Table 1.

TABLE 1
Polypeptide Linkers

| Linker | | SEQ |
|---|---|---|
| Name | Amino Acid Sequence | ID NO: |
| L1 | GGSEGKSSGSGSESKSTGGS | 58 |
| L2 | GGGSGGGS | 59 |
| L3 | GGGSGGGSGGGS | 60 |
| L4 | GGGSGGGSGGGSGGGS | 61 |
| L5 | GGGSGGGSGGGSGGGSGGGS | 62 |
| L6 | GGGGSGGGGSGGGGS | 50 |
| L7 | GGGGSGGGGSGGGGSGGGGS | 45 |
| L8 | GGGGSGGGGSGGGGSGGGGSGGGGS | 63 |
| L9 | GSTSGSGKPGSGEGSTKG | 64 |
| L10 | IRPRAIGGSKPRVA | 65 |
| L11 | GKGGSGKGGSGKGGS | 66 |
| L12 | GGKGSGGKGSGGKGS | 67 |
| L13 | GGGKSGGGKSGGGKS | 68 |
| L14 | GKGKSGKGKSGKGKS | 69 |
| L15 | GGGKSGGKGSGKGGS | 70 |
| L16 | GKPGSGKPGSGKPGS | 71 |
| L17 | GKPGSGKPGSGKPGSGKPGS | 72 |
| L18 | GKGKSGKGKSGKGKSGKGKS | 73 |
| L19 | STAGDTHLGGEDFD | 74 |
| L20 | GEGGSGEGGSGEGGS | 75 |
| L21 | GGEGSGGEGSGGEGS | 76 |
| L22 | GEGESGEGESGEGES | 77 |
| L23 | GGGESGGEGSGEGGS | 78 |
| L24 | GEGESGEGESGEGESGEGES | 79 |
| L25 | GSTSGSGKPGSGEGSTKG | 80 |
| L26 | PRGASKSGSASQTGSAPGS | 81 |
| L27 | GTAAAGAGAAGGAAAGAAG | 82 |
| L28 | GTSGSSGSGSGGSGSGGGG | 83 |
| L29 | GKPGSGKPGSGKPGSGKPGS | 84 |
| L30 | GSGS | 85 |
| L31 | APAPAPAPAP | 86 |
| L32 | APAPAPAPAPAPAPAPAPAP | 87 |
| L33 | AEAAAKEAAAKEAAAAKEAAAAKEAAAAKAAA | 88 |

### Transmembrane Region

As used herein, chimeric antigen receptors (CARs) can comprise a transmembrane region/domain. The CAR can be designed to comprise a transmembrane domain that is fused to the extracellular domain of the CAR. It can similarly be fused to the intracellular domain of the CAR. In one embodiment, the transmembrane domain that is naturally associated with one of the domains in a CAR is used. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention can be derived from (i.e. comprise or engineered from), but are not limited to, CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD33, CD37, CD40, CD45, CD64, CD80, CD86, CD134, CD137, CD154, programmed death-1 (PD-1), inducible T cell costimulator (ICOS), lymphocyte function-associated antigen-1 (LFA-1; CD11a and CD18), CD247, CD276 (B7-H3), LIGHT (tumor necrosis factor superfamily member 14; TNFSF14), NKG2C, Ig alpha (CD79a), DAP10, Fc gamma receptor, MHC class I molecule, TNFR, integrin, signaling lymphocytic activation molecule, BTLA, Toll ligand receptor, ICAM-1, CD5, GITR, BAFFR, LIGHT, HVEM (LIGHTR), KIRDS2, SLAMF7, NKp80 (KLRF1), NKp44, NKp30, NKp46, CD19, CD8 alpha, CD8 beta, IL-2R beta, IL-2R gamma, IL-7R alpha, ITGA4, VLA1, CD49a, IA4, CD49D, ITGA6, VLA-6, CD49f, ITGAD, ITGAE, CD103, ITGAL, CD1a, CD1b, CD1c, CD1d, ITGAM, ITGAX, ITGB1, CD29, ITGB2 (CD18), ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, cytokine receptor, activating NK cell receptors, an immunoglobulin protein, or fragments or any combination thereof. In a preferred embodiment, the transmembrane domain is a CD8 transmembrane domain.

### Immune Cells

According to particular aspects, the invention provides cells that are immune cells that comprise the isolated polynucleotides or vectors comprising the isolated polynucleotides comprising the nucleotide sequence encoding the CAR are provided herein. The immune cells comprising the isolated polynucleotides and/or vectors of the invention can be referred to as “engineered immune cells.” Preferably, the engineered immune cells are derived from a human (are of human origin prior to being made recombinant).

The engineered immune cells can, for example, be cells of the lymphoid lineage. Non-limiting examples of cells of the lymphoid lineage can include T cells and Natural Killer (NK) cells. T cells express the T cell receptor (TCR), with most cells expressing α and β chains and a smaller population expressing γ and δ chains. T cells useful as engineered immune cells of the invention can be CD4⁺ or CD8⁺ and can include, but are not limited to, T helper cells (CD4⁺), cytotoxic T cells (also referred to as cytotoxic T lymphocytes, CTL; CD8⁺ cells), and memory T cells, including central memory T cells, stem-like memory T cells, and effector memory T cells, natural killer T cells, mucosal associated invariant T cells, and γδ T cells. Other exemplary immune cells include, but are not limited to, macrophages, antigen presenting cells (APCs), or any immune cell that expresses an inhibitor of a cell-mediated immune response, for example, an immune checkpoint inhibitor pathway receptor (e.g., PD-1). Precursor cells of immune cells that can be used according to the invention, include, hematopoietic stem and/or progenitor cells. Hematopoietic stem and/or progenitor cells can be derived from bone marrow, umbilical cord blood, adult peripheral blood after cytokine mobilization, and the like, by methods known in the art. The immune cells are engineered to recombinantly express the CARs of the invention.

Immune cells and precursor cells thereof can be isolated by methods known in the art, including commercially available methods (see, e.g., Rowland Jones et al., Lymphocytes: A Practical Approach, Oxford University Press, NY (1999)). Sources for immune cells or precursors thereof include, but are not limited to, peripheral blood, umbilical cord blood, bone marrow, or other sources of hematopoietic cells. Various techniques can be employed to separate the cells to isolated or enrich desired immune cells. For instance, negative selection methods can be used to remove cells that are not the desired immune cells. Additionally, positive selection methods can be used to isolate or enrich for the desired immune cells or precursors thereof, or a combination of positive and negative selection methods can be employed. If a particular type of cell is to be isolated, e.g., a particular T cell, various cell surface markers or combinations of markers (e.g., CD3, CD4, CD8, CD34) can be used to separate the cells.

The immune cells or precursor cells thereof can be autologous or non-autologous to the subject to which they are administered in the methods of treatment of the invention. Autologous cells are isolated from the subject to which the engineered immune cells recombinantly expressing the CAR are to be administered. Optionally, the cells can be obtained by leukapheresis, where leukocytes are selectively removed from withdrawn blood, made recombinant, and then retransfused into the donor. Alternatively, allogeneic cells from a non-autologous donor that is not the subject can be used. In the case of a non-autologous donor, the cells are typed and matched for human leukocyte antigen (HLA) to determine the appropriate level of compatibility. For both autologous and non-autologous cells, the cells can optionally be cryopreserved until ready for use.

Various methods for isolating immune cells that can be used for recombinant expression of the CARs of the invention have been described previously, and can be used, including, but not limited to, using peripheral donor lymphocytes (Sadelain et al., Nat. Rev. Cancer 3:35-45 (2003); Morgan et al., Science 314:126-9 (2006)), using lymphocyte cultures derived from tumor infiltrating lymphocytes (TILs) in tumor biopsies (Panelli et al., J. Immunol. 164:495-504 (2000); Panelli et al., J. Immunol. 164:4382-92 (2000)) and using selectively in vitro expanded antigen-specific peripheral blood leukocytes employing artificial antigen-presenting cells (AAPCs) or dendritic cells (Dupont et al., Cancer Res. 65:5417-427 (2005); Papanicolaou et al., Blood 102:2498-505 (2003)). In the case of using stem cells, the cells can be isolated by methods well known in the art (see, e.g., Klug et al., Hematopoietic Stem Cell Protocols, Humana Press, N J (2002); Freshney et al., Culture of Human Stem Cells, John Wiley & Sons (2007)).

According to particular embodiments, the method of making the engineered immune cells comprises transfecting or transducing immune effector cells isolated from an individual such that the immune effector cells express one or more CAR(s) according to embodiments of the invention. Methods of preparing immune cells for immunotherapy are described, e.g., in WO2014/130635, WO2013/176916 and WO2013/176915, which are incorporated herein by reference. Individual steps that can be used for preparing engineered immune cells are disclosed, e.g., in WO2014/039523, WO2014/184741, WO2014/191128, WO2014/184744 and WO2014/184143, which are incorporated herein by reference.

In a particular embodiment, the immune effector cells, such as T cells, are genetically modified with CARs of the invention (e.g., transduced with a viral vector comprising a nucleic acid encoding a CAR) and then are activated and expanded in vitro. In various embodiments, T cells can be activated and expanded before or after genetic modification to express a CAR, using methods as described, for example, in U.S. Pat. Nos. 6,352,694, 6,534,055, 6,905,680, 6,692,964, U.S. Pat. Nos. 5,858,358, 6,887,466, 6,905,681, 7,144,575, 7,067,318, 7,172,869, 7,232,566, 7,175,843, 5,883,223, 6,905,874, 6,797,514, 6,867,041, US2006/121005, which are incorporated herein by reference. T cells can be expanded in vitro or in vivo. Generally, the T cells of the invention can be expanded by contact with a surface having attached thereto an agent that stimulates a CD3/TCR complex-associated signal and a ligand that stimulates a costimulatory molecule on the surface of the T cells. As non-limiting examples, T cell populations can be stimulated as described herein, such as by contact with an anti-CD3 antibody, or antigen-binding fragment thereof, or an anti-CD3 antibody immobilized on a surface, or by contact with a protein kinase C activator (e.g., bryostatin) in conjunction with a calcium ionophore, or by activation of the CAR itself. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include, e.g., an appropriate media (e.g., Minimal Essential Media or RPMI Media 1640 or, X-vivo 5 (Lonza)) that can contain factors necessary for proliferation and viability, including serum (e.g., fetal bovine or human serum), cytokines, such as IL-2, IL-7, IL-15, and/or IL-21, insulin, IFN-γ, GM-CSF, TGFβ and/or any other additives for the growth of cells known to the skilled artisan. In other embodiments, the T cells can be activated and stimulated to proliferate with feeder cells and appropriate antibodies and cytokines using methods such as those described in U.S. Pat. Nos. 6,040,177, 5,827,642, and WO2012129514, which are incorporated herein by reference.

### Antibodies

In a general aspect, the invention relates to isolated monoclonal antibodies or antigen-binding fragments thereof that specifically bind to a polypeptide linker. The polypeptide linker can, for example, be selected from a polypeptide linker provided in Table 1. In certain embodiments, the polypeptide linker is a (G₄S)ₙ linker, wherein n is at least 2.

In another general aspect, the invention relates to isolated bispecific antibodies or antigen-binding fragments thereof. The isolated bispecific antibodies or antigen binding fragments thereof can be engineered to target a tumor associated antigen (TAA) and a polypeptide linker. The polypeptide linker can, for example, be a (G₄S)ₙ linker, wherein n is at least 2. The bispecific antibodies or antigen-binding fragments thereof can also be engineered to target a tumor associated antigen (TAA) and have a non-antigen binding component (e.g., a non-antigen binding single chain variable fragment (scFv), which comprises a polypeptide linker, (e.g., a (G₄S)ₙ linker, wherein n is at least 2).

Methods of making the antibodies, and methods of using the antibodies in concert with CAR-T cells to treat diseases, including cancer, are also provided. The antibodies of the invention possess one or more desirable functional properties, including, but not limited to, high-affinity for a tumor associated antigen (TAA) and/or a (G₄S)ₙ peptide linker, high specificity for a tumor associated antigen (TAA) and/or a (G₄S)ₙ peptide linker, the ability to activate T cell signaling of a CAR-T cell, the ability to induce effector-mediated tumor cell lysis, the ability to stimulate complement-dependent cytotoxicity (CDC), antibody-dependent phagocytosis (ADPC), and/or antibody-dependent cellular-mediated cytotoxicity (ADCC) against cells expressing a tumor associated antigen, the ability to mediate the recruitment of conjugated drugs, and the ability to inhibit tumor growth in subjects and animal models when administered alone or in combination with other anti-cancer therapies.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to a (G₄S)ₙ peptide linker or a tumor associated antigen (TAA) is substantially free of antibodies that do not bind to a (G₄S)ₙ peptide linker or a tumor associated antigen (TAA)). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that may be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′).

As used herein, the term “single-chain antibody” refers to a conventional single-chain antibody in the field, which comprises a heavy chain variable region and a light chain variable region connected by a short peptide of about 5 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antigen binding domain derived from one species of mammal (e.g., mouse, rat, rabbit, etc.) having the desired specificity, affinity, and capability, while the constant regions correspond to the sequences of an antigen binding domain derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecifc antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope (e.g., a tumor associated antigen (TAA)) and a heavy chain variable domain sequence and a light chain variable domain sequence which have binding specificity for a second epitope (e.g., a (G₄S)ₙ linker peptide). In an embodiment, a bispecific antibody comprises a scFv, or fragment thereof, having binding specificity for a first epitope (e.g., a tumor associated antigen (TAA)), and a scFv, or fragment thereof, having binding specificity for a second epitope (e.g., a (G₄S)ₙ linker peptide). In an embodiment, a bispecific antibody comprises a heavy chain variable domain sequence and a light chain variable domain sequence which have binding specificity for a first epitope (e.g., a tumor associated antigen (TAA)) and a heavy chain variable domain sequence and a light chain variable domain sequence which does not have binding specificity for a second antigen (e.g., a non-antigen binding single chain variable fragment (scFv)). In an embodiment, a bispecific antibody comprises a scFv, or fragment thereof, having binding specificity for a first epitope (e.g., a tumor associated antigen (TAA)), and a scFv, or fragment thereof, having binding specificity for a second epitope (e.g., a non-antigen binding single chain variable fragment (scFv)).

As used herein, the term “tumor associated antigen (TAA)” refers to any antigen expressed and capable of being recognized by an antibody capable of binding the TAA. Examples of tumor associated antigens (TAAs) can include, but are not limited to, prostate specific membrane antigen (PSMA), TMEFF2, KLK2, CD70, PD-1, PD-L1, CTLA-4, EGFR, HER-2, CD19, CD20, CD3, mesothelin (MSLN), prostate stem cell antigen (PCSA), B-cell maturation antigen (BCMA or BCM), G-protein coupled receptor family C group 5 member D (GPRCSD), Interleukin-1 receptor accessory protein (IL1RAP), delta-like 3 (DLL3), carbonic anhydrase IX (CAIX), carcinoembryonic antigen (CEA), CD5, CD7, CD10, CD22, CD30, CD33, CD34, CD38, CD41, CD44, CD49f, CD56, CD74, CD123, CD133, CD138, epithelial glycoprotein-2 (EGP 2), epithelial glycoprotein-40 (EGP-40), epithelial adhesion molecule (EpCAM), folate-binding protein (FBP), fetal acetylcholine receptor (AChR), folate receptor a and b (FRa and b), ganglioside G2 (GD2), ganglioside G3 (GD3), epidermal growth factor receptor (EGFR), epidermal growth factor receptor vIII (EGFRvIII), ERB3, ERB4, human telomerase reverse transcriptase (hTERT), interleukin-13 receptor subunit alpha-2 (IL-13Ra2), k-light chain, kinase insert domain receptor (KDR), Lewis A (CA19.9), Lewis Y (LeY), L1 cell adhesion molecule (LICAM), melanoma-associated antigen 1 (melanoma antigen family A1, MAGE-A1), Mucin-16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor receptor (VEGFR), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), chondroitin sulfate proteoglycan-4 (CSPG4), DNAX accessory molecule (DNAM-1), ephrin type A receptor 2 (EpHA2), fibroblast associated protein (FAP), Gp100/HLA-A2, glypican 3 (GPC3), HA-1H, HERK-V, IL-11Ra, latent membrane protein (LMP1), neural cell-adhesion molecule (N-CAM/CD56), and trail receptor (TRAIL R).

As used herein, an antigen binding domain that “specifically binds to a tumor associated antigen (TAA)” refers to an antigen binding domain that binds to a TAA, preferably a human TAA, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system.

As used herein, the term “(G₄S)ₙ linker peptide” refers to a peptide with a GGGGS (SEQ ID NO:89) amino acid motif with “n” being the number of GGGGS motif repeats. A (G₄S)ₙ linker peptide can have at least 2, at least 3, at least 4, at least 5, at least 6, at least 7, at least 8, at least 9, or at least 10 repeats, or any value in between. In certain embodiments, the (G₄S)ₙ linker peptide has at least 2 repeats. The (G₄S)ₙ linker peptide can, for example, comprise an amino acid sequence selected from the group consisting of SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, and SEQ ID NO:55. In a preferred embodiment, the (G₄S)ₙ linker peptide comprises the amino acid sequence of SEQ ID NO:45.

As used herein, an antigen binding domain that “specifically binds to a (G₄S)ₙ linker peptide” refers to an antigen binding domain that binds to a (G₄S)ₙ linker peptide, preferably a (G₄S)₄ linker peptide, with a KD of 1×10⁻⁷ M or less, preferably 1×10⁸ M or less, more preferably 5×10⁻⁹ M or less, 1×10⁻⁹ M or less, 5×10⁻¹⁰ M or less, or 1×10¹⁰ M or less.

As used herein, a “non-antigen binding single chain variable fragment (scFv)” refers to a scFv that does not specifically bind an antigen. The scFv is designed to not bind any potential antigen with a KD of 1×10⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. Non-specific binding of an antigen by the scFv can occur, but generally, the non-specific binding of an antigen occurs with a KD of 1×10⁻³ M or greater.

The smaller the value of the KD of an antibody, the higher affinity that the antibody binds to a target antigen.

According to a particular aspect, provided herein are isolated monoclonal antibodies or antigen-binding fragments thereof that specifically bind a (G₄S)ₙ polypeptide linker, wherein n is at least 2. The monoclonal antibodies or antigen-binding fragments thereof can comprise a heavy chain complementarity determining region 1 (HCDR1), HCDR2, HCDR3, a light chain complementarity determining region 1 (LCDR1), LCDR2, and LCDR3, having the polypeptide sequences of SEQ ID NOs:1, 2, 3, 4, 5, and 6, respectively.

According to another particular aspect, the monoclonal antibody or antigen-binding fragment thereof that specifically binds a (G₄S)ₙ polypeptide linker comprises a heavy chain variable region having a polypeptide sequence at least 95%, at least 96%, at least 97%, at least 98%, at least 99%, or 100% identical to SEQ ID NO:7, or a light chain variable region having a polypeptide sequence at least 95%, at least 96%, at least 97%, at least 98%, at least 99%, or 100% identical to SEQ ID NO:8.

According to another particular aspect, the monoclonal antibody or antigen-binding fragment thereof that specifically binds a (G₄S)ₙ polypeptide linker is a single chain variable fragment (scFv). The scFv can, for example, comprise an amino acid sequence selected from SEQ ID NO:29 or SEQ ID NO:30.

According to a particular aspect, provided herein are isolated bispecific antibodies or antigen-binding fragments thereof comprising a first polypeptide component and a second polypeptide component, wherein (a) the first polypeptide component comprises (i) a first antigen-binding domain that specifically binds a (G₄S)ₙ polypeptide linker, wherein n is at least 2, or (ii) a non-antigen binding single chain variable fragment (scFv) and a (G₄S)ₙ polypeptide linker, wherein n is at least 2; and (b) the second polypeptide component comprises a second antigen-binding domain that specifically binds a tumor associated antigen (TAA), preferably a human TAA.

According to another particular aspect, the first antigen-binding domain comprises a heavy chain complementarity determining region 1 (HCDR1), a HCDR2, a HCDR3, a light chain complementarity determining region 1 (LCDR1), a LCDR2, and a LCDR3 having the polypeptide sequences of SEQ ID NOs:1, 2, 3, 4, 5, and 6, respectively; and the second antigen-binding domain comprises a heavy chain complementarity determining region 1 (HCDR1), a HCDR2, a HCDR3, a light chain complementarity determining region 1 (LCDR1), a LCDR2, and a LCDR3.

According to another particular aspect, the second antigen-binding domain specifically binds prostate specific membrane antigen (PSMA), preferably human PSMA, or transmembrane protein with EGF-like and two follistatin-like domains 2 (TMEFF2), preferably human TMEFF2. The second antigen-binding domain can, for example, comprise a heavy chain complementarity determining region 1 (HCDR1), a HCDR2, a HCDR3, a light chain complementarity determining region having the polypeptide sequences of (a) SEQ ID NOs:19, 20, 21, 22, 23, and 24, respectively; or (b) SEQ ID NOs:92, 93, 94, 95, 96 and 97, respectively.

According to another particular aspect, the first antigen-binding domain comprises a first heavy chain variable region having a polypeptide sequence at least 95%, at least 96%, at least 97%, at least 98%, at least 99%, or 100% identical to SEQ ID NO:7, and a first light chain variable region having a polypeptide sequence at least 95%, at least 96%, at least 97%, at least 98%, at least 99%, or 100% identical to SEQ ID NO:8; and the second antigen-binding domain having a second heavy chain variable region comprising a polypeptide sequence at least 95%, at least 96%, at least 97%, at least 98%, at least 99%, or 100% identical to SEQ ID NO:25 or SEQ ID NO:90, and a second light chain variable region having a polypeptide sequence at least 95%, at least 96%, at least 97%, at least 98%, at least 99%, or 100% identical to SEQ ID NO:26 or SEQ ID NO:91.

According to another particular aspect, the isolated bispecific antibody or antigen-binding fragment thereof comprises the amino acid sequences selected from SEQ ID NO:35 and SEQ ID NO:28, SEQ ID NO:36 and SEQ ID NO:28, SEQ ID NO:37 and SEQ ID NO:27, SEQ ID NO:38 and SEQ ID NO:27, SEQ ID NO: 101 and SEQ ID NO: 28, SEQ ID NO: 102 and SEQ ID NO: 28, SEQ ID NO: 103 and SEQ ID NO: 98, or SEQ ID NO: 104 and SEQ ID NO: 98.

According to another particular aspect, the non-antigen binding scFv comprises a heavy chain complementarity determining region 1 (HCDR1), a HCDR2, a HCDR3, a light chain complementarity determining region 1, a LCDR2, and a LCDR3 having the polypeptide sequences of SEQ ID NOs:11, 12, 13, 14, 15, and 16, respectively.

According to another particular aspect, the non-antigen binding scFv comprises a heavy chain variable region having an amino acid sequence at least 95%, at least 96%, at least 97%, at least 98%, at least 99%, or 100% identical to SEQ ID NO:17, and a light chain variable region having an amino acid sequence at least 95%, at least 96%, at least 97%, at least 98%, at least 99%, or 100% identical to SEQ ID NO:18.

In another general aspect, the invention relates to an isolated polynucleotide comprising a nucleic acid encoding a chimeric antigen receptor (CAR) as disclosed herein, and isolated nucleic acids encoding monoclonal or bispecific antibodies or antigen-binding fragments thereof as disclosed herein. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding CARs, monoclonal antibodies or antigen-binding fragments thereof, and/or bispecific antibodies or antigen-binding fragments thereof of the invention can be altered without changing the amino acid sequences of the proteins.

In another general aspect, the invention relates to a vector comprising an isolated polynucleotide comprising the nucleic acid encoding the CAR as disclosed herein, and a vector comprising an isolated nucleic acid encoding a monoclonal or bispecific antibody or antigen-binding fragment thereof as disclosed herein. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of an antigen binding domain thereof in the cell. Conventional cloning techniques or artificial gene synthesis can be used to generate a recombinant expression vector according to embodiments of the invention.

In another general aspect, the invention relates to a cell transduced with the vector comprising the isolated polynucleotide comprising a nucleic acid encoding a CAR as disclosed herein. The term “transduced” or “transduction” refers to a process by which exogenous nucleic acid is transferred or introduced into the host cell. A “transduced” cell is one which has been transduced with exogenous nucleic acid. The cell includes the primary subject cell and its progeny. In certain embodiments, the cell is a human CAR-T cell, wherein the T cell is engineered to express the CAR of the invention to treat diseases such as cancer. In certain embodiments, the cell is a human CAR-NK cell, wherein the NK cell engineered to express the CAR of the invention is used to treat diseases such as cancer.

In another general aspect, the invention relates to a method of making a CAR-T cell by transducing a T cell with a vector comprising the isolated nucleic acids encoding the CARs as disclosed herein.

In another general aspect, the invention relates to a method of producing the CAR-T cell as disclosed herein. The method comprising culturing T-cells comprising a nucleic acid encoding a chimeric antigen receptor (CAR) as disclosed herein under conditions to produce the CAR-T cell and recovering the CAR-T cell.

In another general aspect, the invention relates to a method of making a CAR-NK cell by transducing a NK cell with a vector comprising the isolated nucleic acids encoding the CARs as disclosed herein.

In another general aspect, the invention relates to a method of producing a CAR-NK cell as disclosed herein. The methods comprising culturing NK cells comprising nucleic acids encoding the chimeric antigen receptor (CAR) as disclosed herein under conditions to produce the CAR-NK cell and recovering the CAR-NK cell.

In another general aspect, the invention relates to a host cell comprising an isolated nucleic acid encoding a monoclonal or bispecific antibody or antigen-binding fragment thereof as disclosed herein. Any host cell known to those skilled in the art in view of the present disclosure can be used for recombinant expression of antibodies or antigen-binding fragments thereof of the invention. In some embodiments, the host cells are E. coli TG1 or BL21 cells (for expression of, e.g., an scFv or Fab antibody), CHO-DG44 or CHO-Kl cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing a monoclonal or bispecific antibody or antigen-binding fragment thereof as disclosed herein, comprising culturing a cell comprising a nucleic acid encoding the monoclonal or bispecific antibody or antigen-binding fragment thereof under conditions to produce a monoclonal or bispecific antibody or antigen-binding fragment thereof as disclosed herein and recovering the antibody or antigen-binding fragment thereof from the cell or cell culture (e.g., from the supernatant). Expressed antibodies or antigen-binding fragments thereof can be harvested from the cells and purified according to conventional techniques known in the art and as described herein.

### Pharmaceutical Compositions

In another general aspect, the invention relates to a pharmaceutical composition comprising an isolated polynucleotide or nucleic acid as disclosed herein (e.g., an isolated polynucleotide encoding a CAR or an isolated nucleic acid encoding a monoclonal or bispecific antibody or antigen-binding fragment thereof), an isolated polypeptide (e.g., an isolated monoclonal or bispecific antibody or antigen-binding fragment thereof, or a CAR) as disclosed herein, a host cell as disclosed herein, and/or an engineered immune cell as disclosed herein and a pharmaceutically acceptable carrier. The term “pharmaceutical composition” as used herein means a product comprising an isolated polynucleotide or nucleic acid as disclosed herein, an isolated polypeptide as disclosed herein, a host cell as disclosed herein, and/or an engineered immune cell as disclosed herein together with a pharmaceutically acceptable carrier. Polynucleotides, polypeptides, host cells, and/or engineered immune cells of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a polynucleotide, polypeptide, host cell, and/or engineered immune cell pharmaceutical composition can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment of the invention, the pH of the formulation is from about 7.0 to about 9.5. In another embodiment of the invention, the pH of the formulation is from about 3.0 to about 7.0.

In another embodiment of the invention, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments of the invention.

In another embodiment of the invention, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments of the invention.

In another embodiment of the invention, the pharmaceutical composition comprises an isotonic agent. Non-limiting examples of isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g. PEG400), and mixtures thereof. Another example of an isotonic agent includes a sugar. Non-limiting examples of sugars may be mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethylcellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments of the invention.

In another embodiment of the invention, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In another embodiment of the invention, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment of the invention, the pharmaceutical composition comprises a stabilizer, wherein said stabilizer is carboxy-/hydroxycellulose and derivates thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments of the invention.

In further embodiments of the invention, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments of the invention.

In a further embodiment of the invention, the pharmaceutical composition comprises one or more protease inhibitors, such as, e.g., EDTA, and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments of the invention.

In another general aspect, the invention relates to a method of producing a pharmaceutical composition comprising a monoclonal or bispecific antibody or antigen-binding fragment thereof as disclosed herein, comprising combining a monoclonal or bispecific antibody or antigen-binding fragment thereof with a pharmaceutically acceptable carrier to obtain the pharmaceutical composition.

In another general aspect, the invention relates to a method of producing a pharmaceutical composition comprising a CAR-T or CAR-NK cell as disclosed herein, comprising combining a CAR-T or CAR-NK cell with a pharmaceutically acceptable carrier to obtain the pharmaceutical composition.

### Methods of Use

In another general aspect, the invention relates to a method of treating a cancer in a subject in need thereof, comprising administering to the subject pharmaceutical compositions comprising the CAR-T cells and/or CAR-NK cells with the bispecific antibodies or antigen-binding fragments thereof as disclosed herein.

The cancer can, for example, be selected from but not limited to, a prostate cancer, a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to embodiments of the invention, the pharmaceutical compositions comprising the CAR-T cell or CAR-NK cell and/or the bispecific antibody or antigen-binding fragment thereof comprises a therapeutically effective amount of the expressed CARs and bispecific antibodies or antigen-binding fragments thereof as disclosed herein. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to CARs and bispecific antibodies, a therapeutically effective amount means an amount of the CAR molecule expressed in the transduced T cell or NK cell in combination with the bispecific antibody or antigen-binding fragment thereof that modulates an immune response in a subject in need thereof. Also, as used herein with reference to CARs, a therapeutically effective amount means an amount of the CAR molecule expressed in the transduced T cell or NK cell in combination with the bispecific antibody or antigen-binding fragment thereof that results in treatment of a disease, disorder, or condition; prevents or slows the progression of the disease, disorder, or condition; or reduces or completely alleviates symptoms associated with the disease, disorder, or condition.

According to particular embodiments, the disease, disorder or condition to be treated is cancer, preferably a cancer selected from the group consisting of a prostate cancer, a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the invention can be administered in any convenient manner known to those skilled in the art. For example, the cells of the invention can be administered to the subject by aerosol inhalation, injection, ingestion, transfusion, implantation, and/or transplantation. The compositions comprising the cells of the invention can be administered transarterially, subcutaneously, intradermally, intratumorally, intranodally, intramedullary, intramuscularly, intrapleurally, by intravenous (i.v.) injection, or intraperitoneally. In certain embodiments, the cells of the invention can be administered with or without lymphodepletion of the subject.

The pharmaceutical compositions comprising cells expressing CARs as disclosed herein can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the invention in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the invention.

The cells of the invention can be administered in any physiologically acceptable vehicle. A cell population comprising cells of the invention can comprise a purified population of cells. Those skilled in the art can readily determine the cells in a cell population using various well known methods. The ranges in purity in cell populations comprising genetically modified cells of the invention can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the invention are generally administered as a dose based on cells per kilogram (cells/kg) of body weight of the subject to which the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the invention are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors individual to each subject.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment of a cancer. For cancer therapy, the provided compositions can be used in combination with another treatment including, but not limited to, a chemotherapy, an anti-CD20 mAb, an anti-TIM-3 mAb, an anti-LAG-3 mAb, an anti-EGFR mAb, an anti-HER-2 mAb, an anti-CD19 mAb, an anti-CD33 mAb, an anti-CD47 mAb, an anti-CD73 mAb, an anti-DLL-3 mAb, an anti-apelin mAb, an anti-TIP-1 mAb, an anti-FOLR1 mAb, an anti-CTLA-4 mAb, an anti-PD-L1 mAb, an anti-PD-1 mAb, other immuno-oncology drugs, an antiangiogenic agent, a radiation therapy, an antibody-drug conjugate (ADC), a targeted therapy, or other anticancer drugs.

According to particular embodiments, the methods of treating cancer in a subject in need thereof comprise administering to the subject the CAR-T cells and/or CAR-NK cells of the invention in combination with a bispecific antibody or antigen-binding fragment thereof as disclosed herein.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

### Kits

In another general aspect, provided herein are kits, unit dosages, and articles of manufacture comprising any of the isolated polynucleotides comprising nucleic acids encoding CARs as described herein, the CARs as disclosed herein, the engineered CAR-T and/or CAR-NK cells as disclosed herein, the monoclonal and/or bispecific antibodies or antigen-binding fragments thereof as disclosed herein, the isolated nucleic acids encoding the monoclonal and/or bispecific antibodies or antigen-binding fragments thereof as disclosed herein, vectors comprising the isolated polynucleotides or nucleic acids as disclosed herein, and pharmaceutical compositions as disclosed herein. In certain embodiments, the kit preferably provides instructions for its use.

In a particular aspect, provided herein are kits comprising (1) an isolated polynucleotide comprising a nucleic acid encoding a CAR as disclosed herein, and (2) an isolated bispecific antibody or antigen-binding fragment thereof as disclosed herein.

In another particular aspect, provided herein are kits comprising (1) an isolated CAR-T and/or CAR-NK cell as disclosed herein, and (2) an isolated bispecific antibody or antigen-binding fragment thereof as disclosed herein.

In another particular aspect, provided herein are kits comprising (1) an isolated polynucleotide comprising a nucleic acid encoding a CAR as disclosed herein, and (2) an isolated nucleic acid encoding a bispecific antibody or antigen-binding fragment thereof as disclosed herein.

In another particular aspect, provided herein are kits comprising (1) an isolated CAR-T and/or CAR-NK cell as disclosed herein, and (2) an isolated nucleic acid encoding a bispecific antibody or antigen-binding fragment thereof as disclosed herein.

In another particular aspect, provided herein are kits comprising pharmaceutical compositions comprising a pharmaceutically acceptable carrier and (1) the isolated polynucleotide comprising a nucleic acid encoding a CAR as disclosed herein or the isolated CAR-T and/or CAR-NK cell as disclosed herein; and (2) the isolated bispecific antibody or antigen-binding fragment thereof or the isolated nucleic acid encoding the bispecific antibody or antigen-binding fragment thereof.

### Embodiments

The invention provides also the following non-limiting embodiments.

Embodiment 1 is an isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequences of:

a. SEQ ID NOs:1, 2, 3, 4, 5, and 6, respectively; wherein the monoclonal antibody or antigen-binding fragment thereof specifically binds a (G₄S)ₙ polypeptide linker, wherein n is at least 2.

Embodiment 2 is the isolated monoclonal antibody or antigen-binding fragment thereof of embodiment 1, comprising a heavy chain variable region having a polypeptide sequence at least 95% identical to SEQ ID NO:7, or a light chain variable region having a polypeptide sequence at least 95% identical to SEQ ID NO:8.

Embodiment 3 is the isolated monoclonal antibody or antigen-binding fragment thereof of embodiment 1 or 2, comprising:

a. a heavy chain variable region having the polypeptide sequence of SEQ ID NO:7, and a light chain variable region having the polypeptide sequence of SEQ ID NO:8.

Embodiment 4 is the isolated monoclonal antibody or antigen-binding fragment thereof of any one of embodiments 1 to 3, wherein the antibody or antigen-binding fragment thereof is chimeric and/or human or humanized.

Embodiment 5 is the isolated monoclonal antibody or antigen-binding fragment thereof of any one of embodiments 1 to 4, wherein the monoclonal antibody or antigen-binding fragment thereof is a single chain variable fragment (scFv).

Embodiment 6 is the isolated monoclonal antibody or antigen-binding fragment thereof of embodiment 5, wherein the scFv comprises the amino acid sequence selected from SEQ ID NO:29 or SEQ ID NO:30.

Embodiment 7 is an isolated nucleic acid encoding the monoclonal antibody or antigen-binding fragment thereof of any one of claims 1 to 6.

Embodiment 8 is an isolated vector comprising the isolated nucleic acid of embodiment 7.

Embodiment 9 is an isolated host cell comprising the vector of embodiment 8.

Embodiment 10 is an isolated bispecific antibody or antigen-binding fragment thereof comprising a first polypeptide component and a second polypeptide component, wherein
a. the first polypeptide component comprises (i) a first antigen-binding domain that specifically binds a (G₄S)ₙ polypeptide linker, wherein n is at least 2, or (ii) a non-antigen binding single chain variable fragment (scFv) and a (G₄S)ₙ polypeptide linker, wherein n is at least 2; and
b. the second polypeptide component comprises a second antigen-binding domain that specifically binds a tumor associated antigen (TAA), preferably a human TAA.

Embodiment 11 is the isolated bispecific antibody or antigen-binding fragment thereof of embodiment 10, wherein
a. the first antigen-binding domain comprises a heavy chain complementarity determining region 1 (HCDR1), a HCDR2, a HCDR3, a light chain complementarity determining region 1 (LCDR1), a LCDR2, and a LCDR3 having the polypeptide sequences of SEQ ID NOs:1, 2, 3, 4, 5, and 6, respectively; and
b. the second antigen-binding domain comprises a heavy chain complementarity determining region 1 (HCDR1), a HCDR2, a HCDR3, a light chain complementarity determining region 1 (LCDR1), a LCDR2, and a LCDR3.

Embodiment 12 is the isolated bispecific antibody or antigen-binding fragment thereof of embodiment 10 or 11, wherein the second antigen-binding domain specifically binds prostate-specific membrane antigen (PSMA), preferably human PSMA, or transmembrane protein with EGF-like and two follistatin-like domains 2 (TMEFF2), preferably human TMEFF2.

Embodiment 13 is the isolated bispecific antibody or antigen-binding fragment thereof of embodiment 12, wherein the second antigen-binding domain comprises a heavy chain complementarity determining region 1 (HCDR1), a HCDR2, a HCDR3, a light chain complementarity determining region having the polypeptide sequences of:
a. SEQ ID NOs:19, 20, 21, 22, 23, and 24, respectively; or
b. SEQ ID NOs:92, 93, 94, 95, 96, and 97, respectively.

Embodiment 14 is the isolated bispecific antibody or antigen-binding fragment thereof of any one of embodiments 11 to 13, wherein:
a. the first antigen-binding domain comprises a first heavy chain variable region having a polypeptide sequence at least 95% identical to SEQ ID NO:7, and a first light chain variable region having a polypeptide sequence at least 95% identical to SEQ ID NO:8; and
b. the second antigen-binding domain having a second heavy chain variable region comprising a polypeptide sequence at least 95% identical to SEQ ID NO:25 or SEQ ID NO:90, and a second light chain variable region having a polypeptide sequence at least 95% identical to SEQ ID NO:26 or SEQ ID NO:91.

Embodiment 15 is the isolated bispecific antibody or antigen-binding fragment thereof of any one of embodiments 11 to 14, wherein:
a. the first antigen-binding domain comprises a first heavy chain variable region having the polypeptide sequence of SEQ ID NO:7, and a first light chain variable region having the polypeptide sequence of SEQ ID NO: 8; and
b. the second antigen-binding domain comprises a second heavy chain variable region having the polypeptide sequence of SEQ ID NO:25 or SEQ ID NO:90, and the second light chain variable region having the polypeptide sequence of SEQ ID NO:26 or SEQ ID NO:91.

Embodiment 16 is the isolated bispecific antibody or antigen-binding fragment thereof of any one of embodiments 10 to 15, wherein the antibody or antigen-binding fragment thereof is chimeric and/or human or humanized.

Embodiment 17 is the isolated bispecific antibody or antigen-binding fragment thereof of any one of embodiments 10 to 16, wherein the bispecific antibody or antigen-binding fragment thereof comprises the amino acid sequences selected from SEQ ID NO:35 and SEQ ID NO:28, SEQ ID NO:36 and SEQ ID NO:28, SEQ ID NO:37 and SEQ ID NO:27, SEQ ID NO:38 and SEQ ID NO:27, SEQ ID NO: 101 and SEQ ID NO: 28, SEQ ID NO: 102 and SEQ ID NO: 28, SEQ ID NO: 103 and SEQ ID NO: 98, or SEQ ID NO: 104 and SEQ ID NO: 98.

Embodiment 18 is the isolated bispecific antibody or antigen-binding fragment thereof of embodiment 10, wherein the non-antigen binding scFv comprises a heavy chain complementarity determining region 1 (HCDR1), a HCDR2, a HCDR3, a light chain complementarity determining region 1, a LCDR2, and a LCDR3 having the polypeptide sequences of SEQ ID NOs:11, 12, 13, 14, 15, and 16, respectively.

Embodiment 19 is the isolated bispecific antibody or antigen-binding fragment thereof of embodiment 18, wherein the non-antigen binding scFv comprises a heavy chain variable region having an amino acid sequence at least 95% identical to SEQ ID NO:17, and a light chain variable region having an amino acid sequence at least 95% identical to SEQ ID NO:18. Embodiment 20 is the isolated bispecific antibody or antigen-binding fragment thereof of embodiment 18 or 19, wherein the non-antigen binding scFv comprises a heavy chain variable region having the amino acid sequence of SEQ ID NO:17, and a light chain variable region having the amino acid sequence of SEQ ID NO:18.

Embodiment 21 is the isolated bispecific antibody or antigen-binding fragment thereof of any one of embodiments 10 or 18 to 20, wherein the (G₄S)ₙ linker peptide comprises an amino acid sequence selected from the group consisting of SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, and SEQ ID NO:55.

Embodiment 22 is the isolated bispecific antibody or antigen-binding fragment thereof of embodiment 21, wherein the (G₄S)ₙ linker peptide comprises the amino acid sequence of SEQ ID NO:45.

Embodiment 23 is an isolated nucleic acid sequence encoding the isolated bispecific antibody or antigen-binding fragment thereof of any one of embodiments 10-22.

Embodiment 24 is an isolated vector comprising the isolated nucleic acid sequence of embodiment 23.

Embodiment 25 is an isolated host cell comprising the isolated vector of embodiment 24.

Embodiment 26 is an isolated polynucleotide comprising a nucleic acid encoding a chimeric antigen receptor (CAR), wherein the CAR comprises:
a. an extracellular domain comprising (1) a non-antigen binding single chain variable fragment (scFv) and a (G₄S)ₙ polypeptide linker or (2) an antigen binding domain that specifically binds a (G₄S)ₙ polypeptide linker;
b. a transmembrane region; and
c. an intracellular signaling domain.

Embodiment 27 is the isolated polynucleotide of embodiment 26, wherein the non-antigen binding scFv comprises a heavy chain complementarity determining region 1 (HCDR1), a HCDR2, a HCDR3, a light chain complementarity determining region 1, a LCDR2, and a LCDR3 having the polypeptide sequences of SEQ ID NOs:11, 12, 13, 14, 15, and 16, respectively.

Embodiment 28 is the isolated polynucleotide of embodiment 26 or 27, wherein the non-antigen binding scFv comprises a heavy chain variable region having an amino acid sequence at least 95% identical to SEQ ID NO:17, and a light chain variable region having an amino acid sequence at least 95% identical to SEQ ID NO:18.

Embodiment 29 is the isolated polynucleotide of any one of embodiments 26 to 28, wherein the non-antigen binding scFv comprises a heavy chain variable region having the amino acid sequence of SEQ ID NO:17, and a light chain variable region having the amino acid sequence of SEQ ID NO:18.

Embodiment 30 is the isolated polynucleotide of any one of embodiments 26 to 29, wherein the non-antigen binding scFv comprises an amino acid sequence selected from SEQ ID NO:33 or SEQ ID NO:34.

Embodiment 31 is the isolated polynucleotide of any one of embodiments 26 to 30, wherein the (G₄S)ₙ linker peptide comprises an amino acid sequence selected from the group consisting of SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, and SEQ ID NO:55.

Embodiment 32 is the isolated polynucleotide of embodiment 31, wherein the (G₄S)ₙ linker peptide comprises the amino acid sequence of SEQ ID NO:45.

Embodiment 33 is the isolated polynucleotide of any one of embodiments 26 to 32, wherein the extracellular domain is a CD8 extracellular domain.

Embodiment 34 is the isolated polynucleotide of embodiment 33, wherein the CD8 extracellular domain comprises the amino acid sequence of SEQ ID NO:41.

Embodiment 35 is the isolated polynucleotide of any one of embodiments 26 to 34, wherein the transmembrane domain is a CD8 transmembrane domain.

Embodiment 36 is the isolated polynucleotide of embodiment 35, wherein the CD8 transmembrane domain comprises the amino acid sequence of SEQ ID NO:42.

Embodiment 37 is the isolated polynucleotide of any one of embodiments 26 to 36, wherein the intracellular signaling domain comprises a CD137 costimulatory domain and CD3ζ activating domain.

Embodiment 38 is the isolated polynucleotide of embodiment 37, wherein the CD137 costimulatory domain comprises the amino acid sequence of SEQ ID NO:43 and CD3ζ activating domain comprises the amino acid sequence of SEQ ID NO:44.

Embodiment 39 is the isolated polynucleotide of any one of embodiments 26 to 38, wherein the CAR comprises an amino acid sequence selected from SEQ ID NO:39 or SEQ ID NO:40.

Embodiment 40 is the isolated polynucleotide of embodiment 26, wherein the antigen binding domain comprises a heavy chain complementarity determining region 1 (HCDR1), HCDR2, HCDR3, a light chain complementarity determining region 1 (LCDR1), LCDR2, and

LCDR3, having the polypeptide sequences of:
a. SEQ ID NOs:1, 2, 3, 4, 5, and 6, respectively;
   wherein the antigen binding domain specifically binds a (G₄S)ₙ polypeptide linker, wherein n is at least 2.

Embodiment 41 is the isolated polynucleotide of embodiment 40, wherein the antigen binding domain comprises a heavy chain variable region having a polypeptide sequence at least 95% identical to SEQ ID NO:7, or a light chain variable region having a polypeptide sequence at least 95% identical to SEQ ID NO:8.

Embodiment 42 is the isolated polynucleotide of embodiment 40 or 41, wherein the antigen binding domain comprises:
a. a heavy chain variable region having the polypeptide sequence of SEQ ID NO:7, and a light chain variable region having the polypeptide sequence of SEQ ID NO:8.

Embodiment 43 is the isolated polynucleotide of any one of embodiments 40 to 42, wherein the antigen binding domain is chimeric and/or human or humanized.

Embodiment 44 is the isolated polynucleotide of any one of embodiments 40 to 43, wherein the antigen binding domain is a single chain variable fragment (scFv).

Embodiment 45 is the isolated polynucleotide of embodiment 44, wherein the scFv comprises the amino acid sequence selected from SEQ ID NO:29 or SEQ ID NO:30.

Embodiment 46 is a chimeric antigen receptor (CAR) encoded by the isolated polynucleotide of any one of embodiments 26 to 45.

Embodiment 47 is an isolated vector comprising the isolated polynucleotide of any one of embodiments 26 to 45.

Embodiment 48 is an isolated host cell comprising the isolated vector of embodiment 47. Embodiment 49 is the host cell of embodiment 48, wherein the host cell is a T cell, preferably a human T cell.

Embodiment 50 is the host cell of embodiment 48, wherein the host cell is a NK cell, preferably a human NK cell.

Embodiment 51 is a method of producing a chimeric antigen receptor (CAR)-T cell, the method comprising culturing T cells comprising the isolated polynucleotide of any one of embodiments 26 to 45 under conditions to produce a CAR-T cell and recovering the CAR-T cell.

Embodiment 52 is a method of producing a chimeric antigen receptor (CAR)-NK cell, the method comprising culturing NK cells comprising the isolated polynucleotide of any one of embodiments 26 to 45 under conditions to produce a CAR-NK cell and recovering the CAR-NK cell.

Embodiment 53 is a method of making a host cell expressing a chimeric antigen receptor (CAR), the method comprising transducing a T cell or an NK cell with the vector of embodiment 47.

Embodiment 54 is a kit comprising:
a. an isolated polynucleotide comprising a nucleic acid encoding a chimeric antigen receptor (CAR), wherein the CAR comprises:
   i. an extracellular domain comprising (1) a non-antigen binding single chain variable fragment (scFv) and a (G₄S)ₙ polypeptide linker or (2) an antigen binding domain that specifically binds a (G₄S)ₙ polypeptide linker;
   ii. a transmembrane region; and
   iii. an intracellular signaling domain; and
b. the isolated bispecific antibody or antigen-binding fragment thereof of any one of embodiments 10 to 22.

Embodiment 55 is the kit of embodiment 54, wherein the non-antigen binding scFv comprises a heavy chain complementarity determining region 1 (HCDR1), a HCDR2, a HCDR3, a light chain complementarity determining region 1, a LCDR2, and a LCDR3 having the polypeptide sequences of SEQ ID NOs:11, 12, 13, 14, 15, and 16, respectively

Embodiment 56 is the kit of embodiment 54 or 55, wherein the non-antigen binding scFv comprises a heavy chain variable region having an amino acid sequence at least 95% identical to SEQ ID NO:17, and a light chain variable region having an amino acid sequence at least 95% identical to SEQ ID NO:18.

Embodiment 57 is the kit of any one of embodiments 54 to 56, wherein the non-antigen binding scFv comprises a heavy chain variable region having the amino acid sequence of SEQ ID NO:17, and a light chain variable region having the amino acid sequence of SEQ ID NO:18.

Embodiment 58 is the kit of any one of embodiments 54 to 57, wherein the non-antigen binding scFv comprises an amino acid sequence selected from SEQ ID NO:33 or SEQ ID NO:34.

Embodiment 59 is the kit of any one of embodiments 54 to 58, wherein the (G₄S)ₙ linker peptide comprises an amino acid sequence selected from the group consisting of SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, and SEQ ID NO:55.

Embodiment 60 is the kit of embodiment 59, wherein the (G₄S)ₙ linker peptide comprises the amino acid sequence of SEQ ID NO:45.

Embodiment 61 is the kit of any one of embodiments 54 to 60, wherein the CAR comprises an amino acid sequence selected from SEQ ID NO:39 or SEQ ID NO:40.

Embodiment 62 is the kit of embodiment 54, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
a. SEQ ID NOs:1, 2, 3, 4, 5, and 6, respectively;
   wherein the antigen binding domain specifically binds a (G₄S)ₙ polypeptide linker, wherein n is at least 2.

Embodiment 63 is the kit of embodiment 62, wherein the antigen binding domain comprises a heavy chain variable region having a polypeptide sequence at least 95% identical to SEQ ID NO:7, or a light chain variable region having a polypeptide sequence at least 95% identical to SEQ ID NO:8.

Embodiment 64 is the kit of embodiment 62 or 63, wherein the antigen binding domain comprises:
a. a heavy chain variable region having the polypeptide sequence of SEQ ID NO:7, and a light chain variable region having the polypeptide sequence of SEQ ID NO:8.

Embodiment 65 is the kit of any one of embodiments 62 to 64, wherein the antigen binding domain is chimeric and/or human or humanized.

Embodiment 66 is the kit of any one of embodiments 62 to 65, wherein the antigen binding domain is a single chain variable fragment (scFv).

Embodiment 67 is the kit of embodiment 66, wherein the scFv comprises an amino acid sequence selected from SEQ ID NO:29 or SEQ ID NO:30.

Embodiment 68 is a method of treating a cancer expressing a tumor associated antigen (TAA) in a subject in need thereof, the method comprising administering to the subject the isolated host cell of embodiment 48 and a pharmaceutical composition comprising a bispecific antibody or antigen-binding fragment thereof and a pharmaceutically acceptable carrier, wherein the bispecific antibody or antigen binding fragment thereof comprises a first polypeptide component and a second polypeptide component, wherein
a. the first polypeptide component comprises (i) a first antigen-binding domain that specifically binds a (G₄S)ₙ polypeptide linker, wherein n is at least 2, or (ii) a non-antigen binding single chain variable fragment (scFv) and a (G₄S)ₙ polypeptide linker, wherein n is at least 2; and
b. the second polypeptide component comprises a second antigen-binding domain that specifically binds a tumor associated antigen (TAA), preferably a human TAA.

Embodiment 69 is the method of embodiment 68, wherein the bispecific antibody or antigen-binding fragment thereof, wherein:
a. the first antigen-binding domain comprises a heavy chain complementarity determining region 1 (HCDR1), a HCDR2, a HCDR3, a light chain complementarity determining region 1 (LCDR1), a LCDR2, and a LCDR3 having the polypeptide sequences of SEQ ID NOs:1, 2, 3, 4, 5, and 6, respectively; and
b. the second antigen-binding domain comprises a heavy chain complementarity determining region 1 (HCDR1), a HCDR2, a HCDR3, a light chain complementarity determining region 1 (LCDR1), a LCDR2, and a LCDR3.

Embodiment 70 is the method of embodiment 68 or 69, wherein the second antigen-binding domain specifically binds prostate-specific membrane antigen (PSMA), preferably human PSMA, or transmembrane protein with EGF-like and two follistatin-like domains 2 (TMEFF2), preferably human TMEFF2.

Embodiment 71 is the method of any one of embodiments 68 to 70, wherein the second antigen-binding domain comprises a heavy chain complementarity determining region 1 (HCDR1), a HCDR2, a HCDR3, a light chain complementarity determining region having the polypeptide sequences of:
a. SEQ ID NOs:19, 20, 21, 22, 23, and 24, respectively; or
b. SEQ ID NOs:92, 93, 94, 95, 96, and 97, respectively.

Embodiment 72 is the method of any one of embodiments 68 to 71, wherein:
a. the first antigen-binding domain comprises a first heavy chain variable region having a polypeptide sequence at least 95% identical to SEQ ID NO:7, and a first light chain variable region having a polypeptide sequence at least 95% identical to SEQ ID NO:8; and
b. the second antigen-binding domain comprises a second heavy chain variable region having a polypeptide sequence at least 95% identical to SEQ ID NO:25 or SEQ ID NO:90, and a second light chain variable region having a polypeptide sequence at least 95% identical to SEQ ID NO:26 or SEQ ID NO:91.

Embodiment 73 is the method of any one of embodiments 68 to 72, wherein:
a. the first antigen-binding domain comprises a first heavy chain variable region having the polypeptide sequence of SEQ ID NO:7, and a first light chain variable region having the polypeptide sequence of SEQ ID NO: 8; and
b. the second antigen-binding domain comprises a second heavy chain variable region having the polypeptide sequence of SEQ ID NO:25 or SEQ ID NO:90, and a second light chain variable region having the polypeptide sequence of SEQ ID NO:26 or SEQ ID NO:91.

Embodiment 74 is the method of embodiments 68 to 73, wherein the bispecific antibody or antigen-binding fragment thereof is chimeric and/or human or humanized.

Embodiment 75 is method of any one of embodiments 68 to 74, wherein the bispecific antibody or antigen-binding fragment thereof comprises the amino acid sequences selected from SEQ ID NO:35 and SEQ ID NO:28, SEQ ID NO:36 and SEQ ID NO:28, SEQ ID NO:37 and SEQ ID NO:27, SEQ ID NO:38 and SEQ ID NO:27, SEQ ID NO: 101 and SEQ ID NO: 28, SEQ ID NO: 102 and SEQ ID NO: 28, SEQ ID NO: 103 and SEQ ID NO: 98, or SEQ ID NO: 104 and SEQ ID NO: 98.

Embodiment 76 is the method of embodiment 68, wherein the non-antigen binding scFv comprises a heavy chain complementarity determining region 1 (HCDR1), a HCDR2, a HCDR3, a light chain complementarity determining region 1, a LCDR2, and a LCDR3 having the polypeptide sequences of SEQ ID NOs:11, 12, 13, 14, 15, and 16, respectively.

Embodiment 77 is the method of embodiment 76, wherein the non-antigen binding scFv comprises a heavy chain variable region having an amino acid sequence at least 95% identical to SEQ ID NO:17, and a light chain variable region having an amino acid sequence at least 95% identical to SEQ ID NO:18.

Embodiment 78 is the method of embodiment 76 or 77, wherein the non-antigen binding scFv comprises a heavy chain variable region having the amino acid sequence of SEQ ID NO:17, and a light chain variable region having the amino acid sequence of SEQ ID NO:18. Embodiment 79 is the method of embodiments 68 or 76 to 78, wherein the (G₄S)ₙ linker peptide comprises an amino acid sequence selected from the group consisting of SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, and SEQ ID NO:55.

Embodiment 80 is the method of embodiment 79, wherein the (G₄S)ₙ linker peptide comprises the amino acid sequence of SEQ ID NO:45.

### EXAMPLES

### Example 1: Development of Universally Recognized Chimeric Antigen Receptor (CAR) Domain

### Materials and Methods

### Bispecific Cloning

Bispecific mabs targeting two different prostate cancer antigens were generated: (a) anti-G₄S×anti-PSMA and (b) anti-G₄S×anti-TMEFF2. DNA gBlocks were synthesized containing the sequence of anti-G₄S scFv or anti-PSMA scFv or anti-TMEFF2 scFv. The designed heavy chain molecules were synthesized into gblocks (IDT; Coralville, Iowa) containing 15 bp overlaps at the 5′ and 3′ ends for ligation independent cloning using InFusion method (ClonTech (Takara); Mountain View, Calif.). All light chain constructs were inserted into pLonza vector containing the BswiI and HindIII restriction sites for in-frame ligation to the human kappa constant domain. Human CD4 signal peptides were encoded to allow for efficient secretion of mAbs into culture supernatant. All gblocks were reconstituted in sterile water and incubated at 50° C. for 10 minutes as per manufacturer protocol. pLonza vector (Lonza; Basel, Switzerland) was linearized using EcoRI and HindIII followed by gel extraction and cleanup. A 2:1 mass ratio of linearized vector to insert was used followed by heat pulse at 50° C. for 15 minutes. The infusion reactions were transformed into Stellar competent cells (ClonTech) and resultant colonies were scaled for miniprep. All constructs were sequence verified and scaled up using Endotoxin free maxi preparation kits (Qiagen; Hilden, Germany).

### CAR-T Cloning, Lentiviral Production, and CAR-T Generation

H3-23/L1-39, a germline scFv (Teplyakov et al., MAbs 8:1045-63 (2016)), for a CAR-T construct was designed to include 5′ and 3′ overlap corresponding to the EcoRI and SpeI restrictions sites in a lentiviral vector. The designed DNA inserts were codon optimized for Homo sapiens and synthesized at IDT. Cloning of constructs was performed using InFusion method described above. All constructs were sequence confirmed prior to transfection.

The scFv against G₄S linker was generated. Human-codon optimized DNA comprising the CD8α-chain signal sequence, scFv sequence, CD8 α hinge and transmembrane domains, 4-1BB, and CD3 domain were cloned into the lentiviral vector. In order to produce high-titer replication-defective lentiviral vectors, 293 T human embryonic kidney cells were transfected with pVSV-G, pRSV.REV, pMDLg and CAR-containing lentiviral vector using lipofectamine 2000 (Invitrogen; Carlsbad, Calif.). The viral supernatant was harvested at 24 and 48 hours post-transfection. Viral particles were concentrated using Lenti-X concentrator (Takara; Mountain View, Calif.). Concentrated viral particles were resuspended in PBS, and stored frozen at −80° C. Primary human CD4+ and CD8+ T cells were isolated from healthy volunteer donors following leukapheresis by negative selection, and purchased from HemaCare. T cells were cultured in complete media (RPMI 1640 supplemented with 10% heat inactivated fetal bovine serum (FBS), 100 U/ml penicillin, 10-mM HEPES), stimulated with anti-CD3 and anti-CD28 mAbs coated beads (Invitrogen). 24 hr after activation, T-cells were transduced with lentiviral vector at MOI of ˜5-10. Human recombinant interleukin-2 (IL-2; Peprotech; Rocky Hill, N.J.) was added every other day to 50 IU/ml final concentration and 0.5-1×10⁶ cells/ml cell density was maintained. CAR surface expression was verified by flow cytometry using mAb against G₄S linker as primary staining following PE-labeled anti-human Fc antibody as secondary staining.

### Expression

ExpiCHO mammalian expression system was used for protein expression (Invitrogen; Carlsbad, Calif.). To ensure proper light chain loading in the mature protein, a 3:1 light chain:heavy chain DNA ratio was used. Cells were grown to a density of 6×10⁶ cells/ml and split prior to transfection. The bispecific and monospecific antibodies were expressed and produced by co-transfection of light chain and heavy chain (as shown in Table 2). The DNA mixture was incubated with Expifectamine and immediately added to the culture. ExpiCHO suspension cultures were harvested by centrifuging at 3000 g for 10 minutes to pellet cells. The supernatant was filtered using 0.22 μm membrane to remove residual cellular particulates. Roche Complete protease inhibitors were added to the supernatant to minimize proteolytic degradation. The supernatants were stored at 4° C. until purification.

TABLE 2
Sequences required for making bispecific and monospecific antibodies

| | | Heavy chain | Light chain |
|---|---|---|---|
| Antibody | Sample name | (SEQ ID NO) | (SEQ ID NO) |
| Bispecific | BiSpAb1 | PTCB330 (36) | CEN-63-13-VK (28) |
| antibody | BiSpAb2 | MSCB336 (35) | CEN-63-13-VK (28) |
| | BiSpAb3 | MSCB337 (37) | PS3B35 PSMA VK (27) |
| | BiSpAb4 | MSCB338 (38) | PS3B35 PSMA VK (27) |
| | BiSpAb5 | MSCB542 (101) | CEN-63-13-VK (28) |
| | BiSpAb6 | MSCB543 (102) | CEN-63-13-VK (28) |
| | BiSpAb7 | MSCB544 (103) | TMEB570 TMEFF2 VK (98) |
| | BiSpAb8 | MSCB545 (104) | TMEB570 TMEFF2 VK (98) |
| Monospecific | CEN-63-13 mAb | CEN-63-13-VH (7) | CEN-63-13-VK (28) |
| antibody | | | |

### Purification of Recombinant Proteins

5 ml HiTrap MabSelect Sure (GE Healthcare; Chicago, Ill.) columns were equilibrated in PBS pH 7.4. Supernatants were applied to the column at a flow rate of 1 mL/min for maximum capture. Columns were washed using 20 column volumes of PBS until a clean baseline was obtained as monitored by UV A₂₈₀. Isocratic elution was performed with 10 column volumes of 100 mM Na Citrate, pH 3.5. The eluted protein was fractionated and absorbance at A₂₈₀ was used to determine concentration. Fractions were tested for the presence of recombinant protein using non-denaturing and denaturing SDS PAGE gels (BioRad; Hercules, Calif.) and pooled. Proteins purified in this manner were deemed >95% pure by SDS PAGE analysis and were stored at 4° C. until use.

### Human T Cells Culture and Electroporation

Human PanT cells were isolated from the peripheral blood monocyte cells (PBMC) of healthy donors and were cultured in complete T cell media/RPMI media with 10% FCS, 2 mM GlutaMax, 1 mM sodium pyruvate, 55 μM β-mercaptoethanol and 100 U penicillin/streptomycin.

PanT cells were expanded ex vivo using magnetic Dynabeads of anti-CD3/CD28 for about 12-14 days following manufacturer protocol (ThermoFisher; Waltham, Mass.). These cells were frozen at 1×10⁶ cells/vial and stored in liquid nitrogen.

Prior to electroporation, T cells were pre-activated by Dynabeads with 10 ng/ml recombinant human IL-2 for 24 hours. 5-10×10⁶ T cells were resuspended in 204 primary cell nucleofection solution (P3 primary cell 4D-Nucleofector kit). T cells were mixed with 10 μg IVT RNA and transferred to Nucleofection cuvette strips. Cells were electroporated using a 4D nucleofector (Lonza) using the program EO105 for activated human T cells. After electroporation, prepared T cell media was used to transfer transfected cells in 96-well plate and continued to culture for 3-4 days.

### T Cell Activation and Cytokine Profiling

A high throughput assay of TCA (T cell activation assay) was performed using the T cell activation cell and cytokine profiling kit in purpose to identify T-cell subsets and measure T-cell activation and cytokine secretion. Briefly, tumor cell line PC3 M11 was cultured in 96 well plate (1×10⁴ cell/well). CAR-T cells were added the next day at a concentration of 5×10⁴ cells (E:T=5:1). Bi-specific molecules were added into the co-culture wells with final concentration of 504. Cells were co-cultured for 32 hours. At the end of co-culture, cells were washed once, and were stained with antibody against CD69 and CD25 and HLA-DR. Further, the levels of secreted cytokines were quantitated including IFN-γ and TNFα and IL-2 and IL-6 and IL-17 and IL-13 and IL-10 and GM-CSF. Data were acquired on the Intellicyt iQue Plus and analyzed with ForeCyt software using the T cells activation kit data template.

### ELISA

MaxiSorb 96 plates (Nunc; Roskilde, Denmark) were coated with antigen at 0.1 μg/mL concentration and incubated overnight at 4° C. Plates were washed 3 times with PBS and blocked with PBS plus 5% milk powder for 1 hour at room temperature. Plates were washed 3 times with PBS. Serial dilutions (1:3) of the CEN-63-13 mAb and control mAb at starting concentrations of 100 nM were prepared in PBS and added to the wells of the plate for room temperature incubation for 90 minutes. Plates were washed 3 times with PBS. Following the wash step, 1004 of 1:10000 Goat Anti-Human Fc Horse Radish Peroxidase detection antibody was added and incubated for 1 hour at room temperature. Plates were washed 3 times with PBS and 1004 of TMB substrate (1-step TMB, Thermo Fisher) was added at 1:100 dilution. Optical density at 450 nm was measured on (SpectraMax M5, Molecular Devices; San Jose, Calif.). Data was analyzed in GraphPad Prism software (Graphpad).

Flow Cytometry Fluorescence Detection of G4S Peptide Containing scFvs

HEK293-T cells were cultured in standard DMEM with (Dulbecco's Modified Eagle's Medium Components comprising glucose, L-glutamine, NaHCO₃, and phenol red). Transfection of cells with mRNA for linker containing scFv protein constructs was carried out following manufacturer's protocol (MessengerMax, Invitrogen). 5 μg of IVT synthesized mRNA was transfected in HEK293-T cells at a density of 1×10⁶ cells/mL and incubated 24 hours prior to flow cytometry analysis. Cells were added to 96 well U bottom plates at a concentration of 100,000 cells/well. Plates were spun at 300 g for 3 minutes and supernatant was discarded. Sytox green (Invitrogen) Live/Dead stain was added to the cells and incubated for 10 minutes at room temperature in a dark chamber. The cells were washed twice with PBS, and the supernatant was discarded. A 12 point 1:3 serial dilution with a starting concentration of 100 nM of primary antibody was prepared. The dilution series was added to cells and incubated for 1 hour at 4° C. in the dark. Plates were spun at 300 g for 3 minutes, the supernatants discarded, and cells washed twice with FACS running buffer (Becton Dickinson (BD), Franklin Lakes, N.J.). Secondary antibody (Anti human Fc, Biolegend; San Diego, Calif.) was diluted in FACS buffer according to manufacturer's protocol. 50 μl of secondary antibody was added to the cells and incubated for 30 minutes at 4° C. in the dark. Cells were then washed twice with FACS buffer, supernatants discarded, and then reconstituted in 504 of FACS buffer. The Intellicyt iQue Screener Plus (Sartorius) was used to detect cell surface binding of anti-G₄S antibodies. Data processing was performed using ForeCyt software (Sartorius; Gottingen, Germany). Dose response binding curves were generated using GraphPad Prism 7 software (GraphPad).

### Determination of Binding Epitope by BioLayer Interferometry

The ForteBioOctet RED384 system (Pall Corporation; Port Washington, N.Y.) was used to measure binding kinetics between biotinylated G4S peptides and the rabbit anti-(G₄S)₄ linker antibody. Biotinylated G4S peptides (WT, control or truncation peptides) were immobilized on streptavidin sensors, and rabbit anti-(G₄S)₄ linker antibody was tested for binding to sensor-immobilized G4S peptides according to manufacturer's instructions. Association and dissociation rates were measured by the shift in wavelength (nm) and KD (equilibrium dissociation constant) was obtained by fitting the data to 1:1 binding model. All reactions were performed at 25° C. in 1× kinetics buffer (ForteBio; Fremont, Calif.). Data were collected with Octet Data Acquisition program (ForteBio) and analyzed using Octet Data Analysis program (ForteBio).

### CAR-T Cells CD107a Assay and Proliferation Assay

For CD107a assay, CAR-T cells were co-cultured with PC3 prostate tumor cells in 96-well plate at an effector to target ratio (E:T) equal to 5:1 in the presence or absence of anti-PSMA×anti-G4S BsAbs (5 mg/ml). Phycoerythrin-labeled anti-CD107a antibody was added 1 hour before adding Golgi Stop (BD Bioscience; San Jose, Calif.) and the plate was incubated for 3 hours. The anti-CD8 antibody were added and incubated at 37° C. for 30 minutes. After incubation, the samples were washed once and subjected to flow cytometry. The data were analyzed by FlowJo software. For T cell proliferation assay, CAR-T cells were pre-labeled with 5 mM CFSE (Invitrogen) according to the manufacturer's protocol. CAR-T cells were cocultured with PC3 prostate tumor cells at an effector to target cells ratio (E:T ratio) of 1 to 1 in 96-well round bottom plate in 200 μl RPMI complete media. The BsAbs of anti-PSMA×anti-G₄S (5 mg/ml) was added. After a 3-day incubation, T cells were stained with anti-CD3 mAb and analyzed for CFSE distribution.

### Cytotoxicity Assays by Xcelligence

Cytotoxicity was measured in a real-time cell analyzer xCelligence (Roche; Basel, Switzerland) using adherent tumor cell lines as target cells. All experiments were performed using the respective target cell culturing media. 50-μl of medium was added to E-Plates 96 (Roche, Grenzach-Wyhlen, Germany) for measurement of background values. Target cells used in the experiments include PC3M11 and C4-2B and LnCap tumor cell lines. Target cells were seeded in an additional 100 μl medium at a density of around 10,000 cells per well. Suitable cell densities were determined by previous titration experiments. Cell attachment was monitored using the RTCA SP (Roche) instrument and the RTCA software Version 1.1 (Roche) until the plateau phase was reached.

CAR-T cells were added at different effector to target ratios (E:T) ranging from 20:1 to 1:1, or variant dosages of BsAb were added at concentrations ranging from 0.2 to 20 mg/ml. Upon addition of effector cells, impedance measurements were performed every 15 minutes for up to 81 hours. All experiments were performed in triplicates. Changes in electrical impedance were expressed as a dimensionless cell index (CI) value, which derives from relative impedance changes corresponding to cellular coverage of the electrode sensors, normalized to baseline impedance values with medium only. To analyze the acquired data, CI values were exported, and percentage of lysis was calculated in relation to the control cells lacking any effector T cells. The percentage of cytolysis is readily calculated using a simple formula: Percentage of cytolysis=((Cell Index_{no effector}−Cell Index_{effector})/Cell Index_{no effector})×100.

Cytotoxicity of the CAR-expressing T cells was also tested by using the IncuCyte zoom living cell imaging system. Co-culture was set up the same as the above in xcelligence assay. Images were taken every 30 minutes and the number of dead cells was quantified.

Cytokine Assay (Intellicyt iQue)

The intellicyt human T cell activation and cytokine profiling kit was applied for T cell activation and cytokine profile. Briefly, CAR-T cells were co-cultured with PC3 prostate tumor cells at an effector to target cells ratio (E:T ratio) of 1 to 1 in 96-well round bottom plate in 200 μl RPMI complete media. The BsAbs of anti-PSMA×anti-G₄S (5 mg/ml) was added. Co-culture without BsAb were used as control. 24 hours later, T cell activation was assessed by the TCA kit from a 30 μl cell/supernatant mixture sample following the protocol. Samples were acquired on the Intellicyt iQue Screener PLUS. Standard curves to quantitate the levels of secreted cytokines. Data were analyzed with ForeCyt software.

### Results

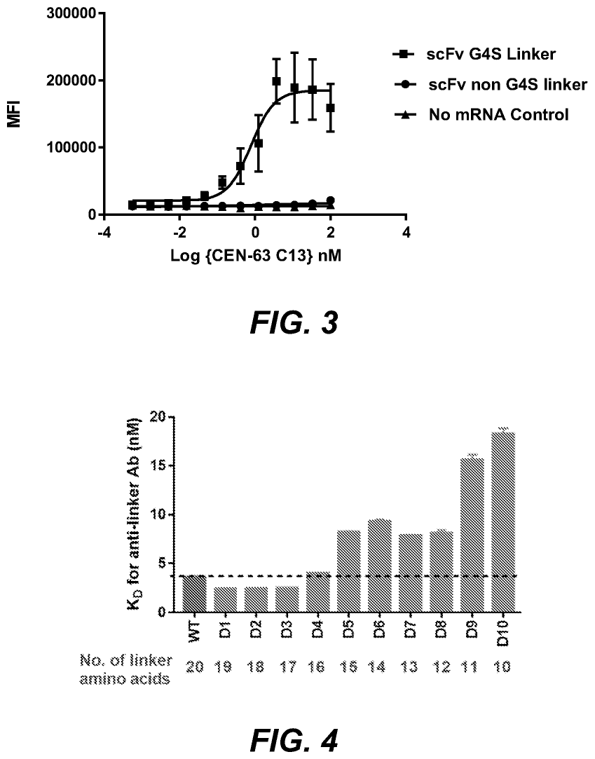

To develop a modular T cell therapy, a CAR stalk was designed that contained a peptide that would be universally recognized by an antibody. The (G₄S)₄ (SEQ ID NO:45) linker peptide was chosen because of its relatively good biophysical properties. In order to obtain monoclonal antibodies against G₄S, rabbits were immunized with the G₄S peptide. Following the generation of an immune response, the spleens from these rabbits were harvested. V gene recovery of the variable heavy and light regions was performed. Expression of the v regions on a human IgG1 backbone with human kappa light chains was followed by 1 step affinity chromatography. ELISA and flow cytometry assays confirmed that one monoclonal antibody, CEN-63-13, bound immunospecifically to the G₄S linker with a dissociation constant of 0.57 nM (FIGS. 2 and 3).

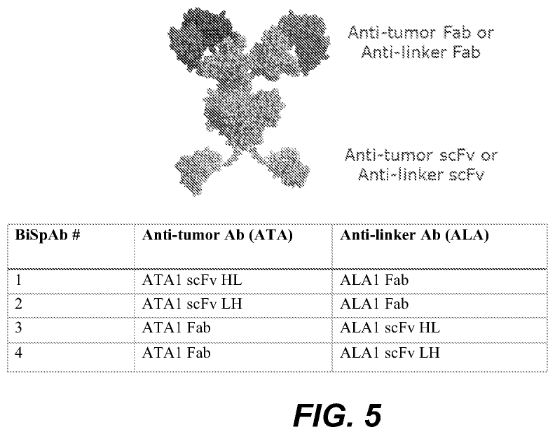

The CEN-63-13 variable regions were reformatted into single chain Fragment variable (scFv)s in both the variable heavy/linker/variable light (HL) (SEQ ID NO:29) and the variable light/linker/variable heavy (LH) (SEQ ID NO:30) orientations. Using previously discovered variable region sequences against PSMA (Clone ID: PS3B35) (Chang et al., Cancer Res. 59(13):3192-8 (1999), four (4) different Morrison scaffold bispecific antibody constructs were designed (FIG. 5) (Table 2). To test the expression and stability of the reformatted scFvs, Morrison constructs with anti-PSMA Fab domains with both HL and LH scFvs (SEQ ID NO:31 and 32, respectively) fused to the C terminus of HuIgG1 were designed. A 9 amino acid linker, (GAG)₃ (SEQ ID NO:56) was used as a tether between the scFvs and the Fc region. Next, CEN-63-13 variable region Fabs with anti-PSMA scFvs fused to the C terminus of human IgG1 in the both HL and LH orientations were designed. Each of these constructs was expressed in suspension CHO expression system and was purified by 1 step affinity chromatography. Complementarity determining regions (CDRs) for CEN-63-13 and PS3B35 antibodies are provided in Table 4. Utilizing a similar strategy as described above, similar bispecific conduit antibodies were derived with the tumor associated antigen (TAA) being another prostate cancer TAA, i.e., Transmembrane Protein with EGF Like and Two Follistatin Like Domains (TMEFF2).

The optimal binding epitope for CEN-63-13 variable region was determined utilizing constructs of the G₄S peptide. A panel of truncated peptides was assayed by Bio Layer Interferometry. Peptides missing up to 8 amino acids (SEQ ID NOs:46-53) from the WT G₄S linker (SEQ ID NO:45) only displayed a 2-fold decrease in binding affinity. The 10-mer peptide (SEQ ID NO:55), with a 6-fold decrease in binding affinity compared to WT, represents the smallest linker possible to be detected by CEN-63-13 (FIG. 4). Table 3 shows the K_{D} values for CEN-63-13 binding to protein and peptide antigens as determined using bio-layer interferometry.

TABLE 3
K_{D} values for CEN-63-13 binding to (G₄S)₄ peptide and non-antigen
binding H3-23/L1-39 scFV with (G₄S)₄ peptide linker.

| Anti-linker Ab | Antigen | K_{D} |
|---|---|---|
| CEN-63-13 | H3-23/L1-39 scFv | 8.1 nM |
| | with (G₄S)₄ peptide | |
| | linker | |
| CEN-63-13 | (G₄S)₄ peptide | 3.6 nM |

In order to direct T cells to tumor cells using conduit bispecific antibodies, the G₄S peptide linker (SEQ ID NO:45) was engineered into an “inert” scFv (SEQ ID NO:33 and 34) in a 2^{nd} generation CAR stalk (SEQ ID NO:39 and SEQ ID NO:40).

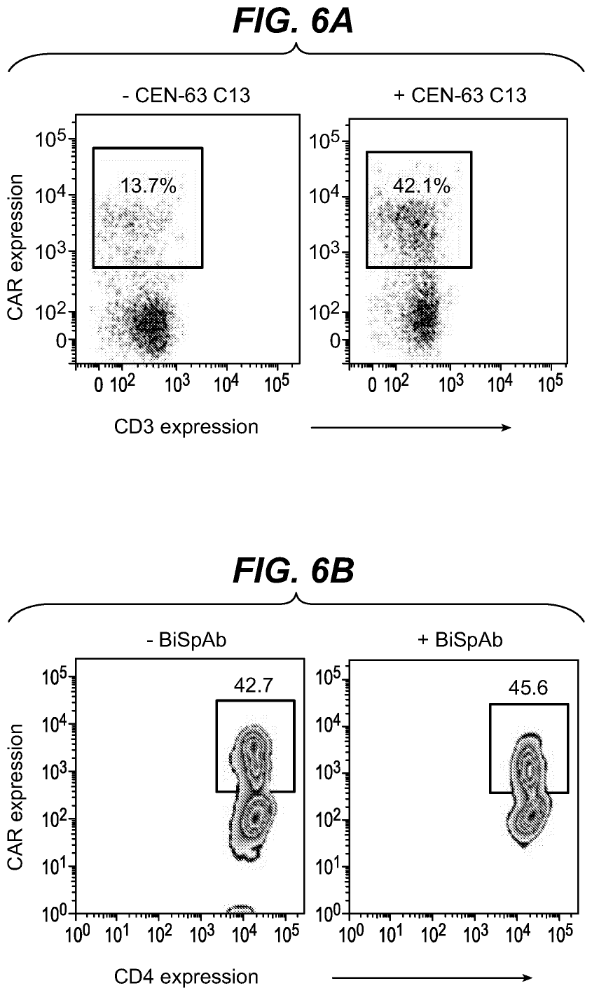

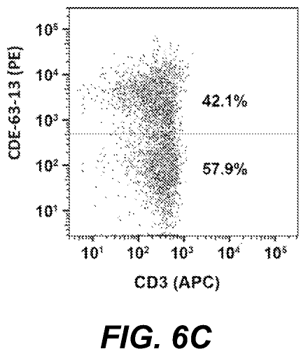

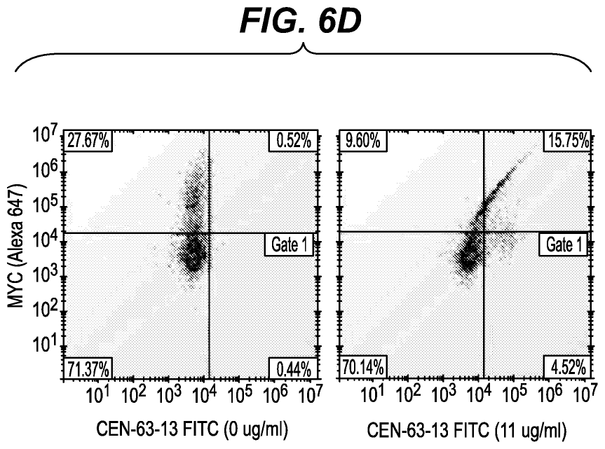

Both Lentiviral transfected Pan-T cells as well as Pan-T cells transfected with CAR encoding mRNA were generated. The extracellular (G₄S)₄ ScFv linker could be detected via flow cytometry analysis utilizing the human CEN-63-13 antibody and a PE-labeled anti-human secondary antibody (FIG. 6C). In addition to binding the (G₄S)₄ containing Isotype CAR, binding was also demonstrated to Pan-T cells that had been transduced via lentivirus expressing an anti-CD19 CAR with a (G₄S)₃ linker and a N-terminal MYC tag. CEN-63-13 co-stains with MYC positive CAR-T cells (FIG. 6D).

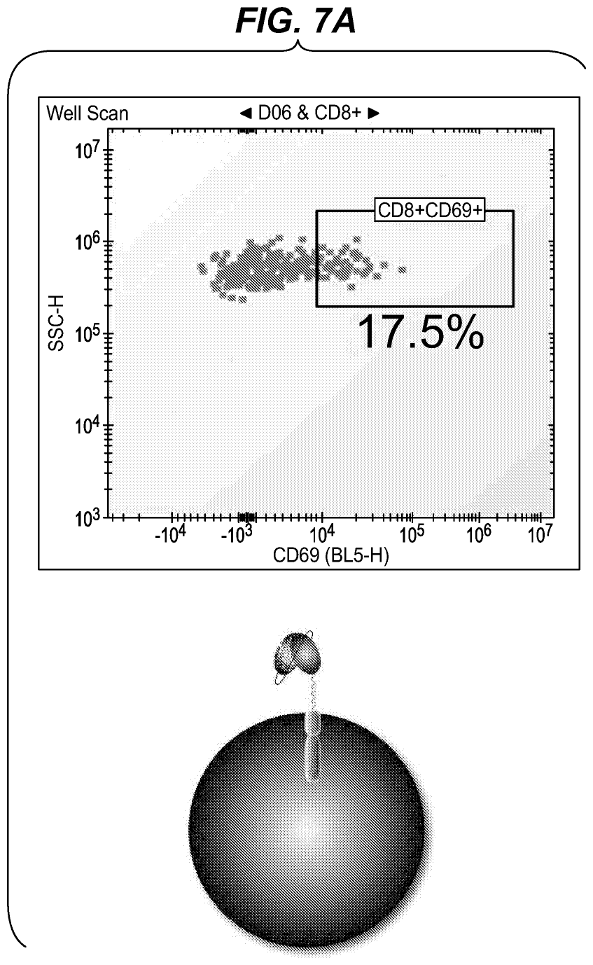

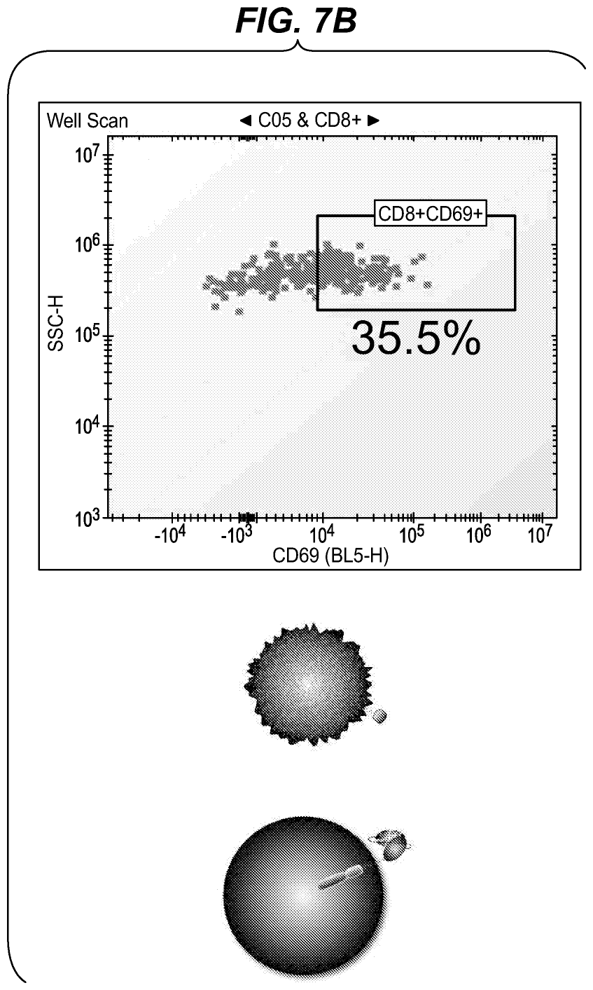

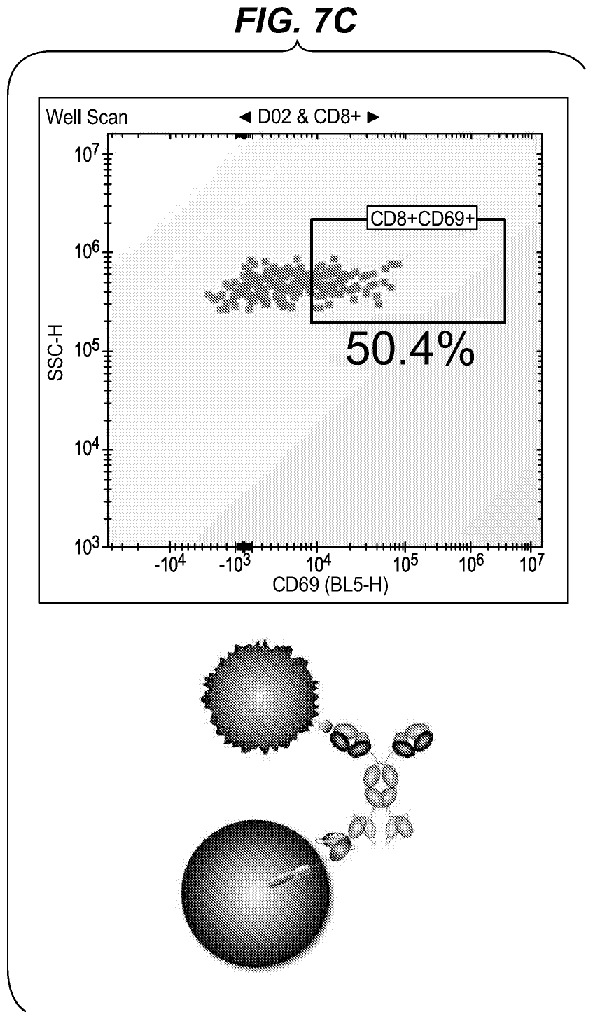

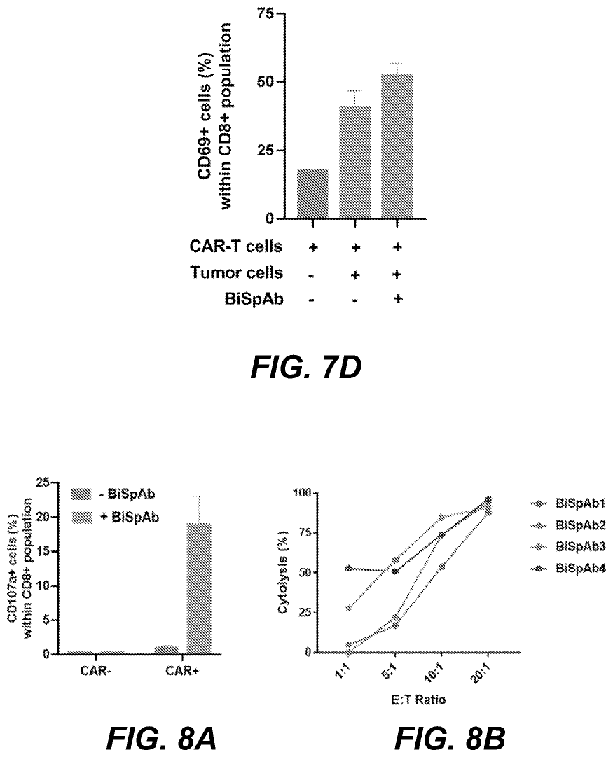

T cells were also transfected with DNA encoding the CAR stalk. A 3-fold increase in CD69 expression compared to T cells only was observed, indicating activation of CAR-T cells (FIGS. 7A-7D).

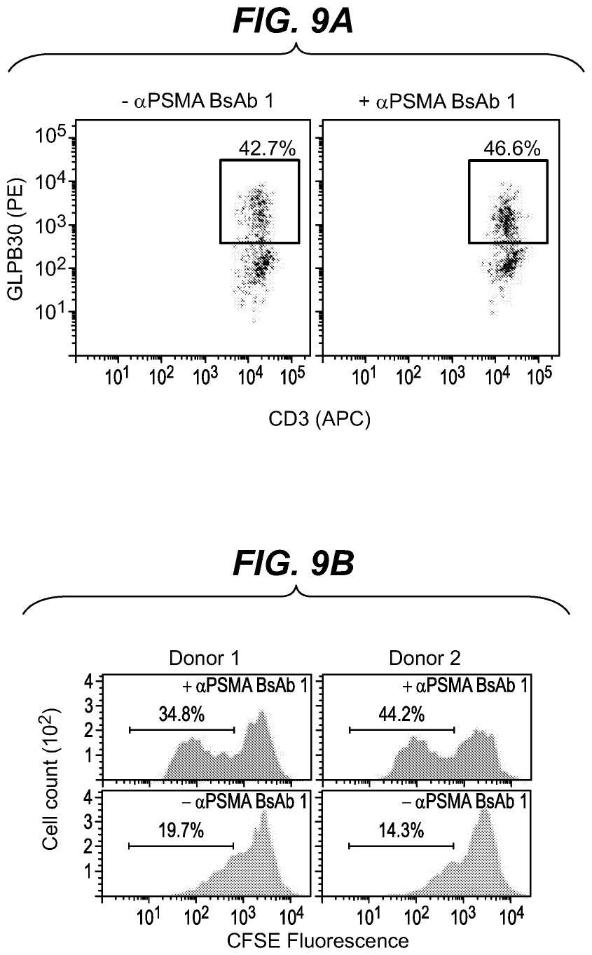

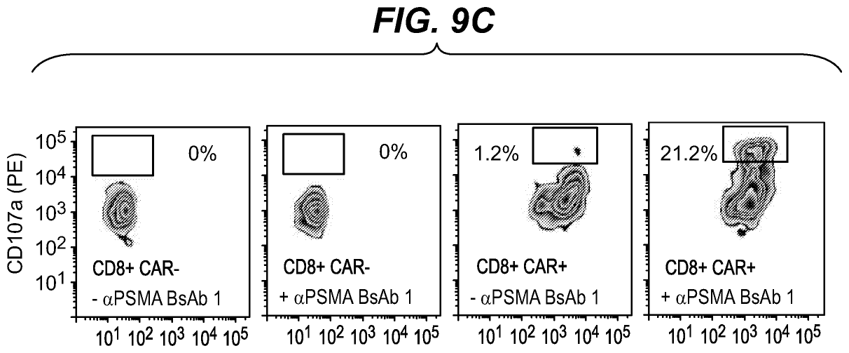

Whether the presence of a bispecific antibody (BsAb) affected CAR surface expression in isotype ScFv expressing CAR-T cells was subsequently examined. Cultured CAR-T cells were divided, and BsAb (5 μg/ml) was added into cultured CAR-T cells while no antibody was added to control wells. Cells were then extensively washed and CAR surface expression was observed at 24 hours. As shown in FIG. 9A, incubation with the BsAb did not alter the surface expression level of CAR.

As incubation with the BsAb alone did not alter CAR proliferation or surface expression, next it was examined whether the addition of tumor cells in addition to the bispecific molecule could induce proliferation. Proliferation of CFSE-labeled T cells in the presence or absence of BsAb with PSMA expressing tumor cells was compared. Proliferation of T cells (observed in two donors) was demonstrated when bispecific antibodies were added into co-culture (FIG. 9B). Notably, some proliferation was observed in the absence of BsAb, but this is likely due to allogeneic reactions with the tumor cells.

CD107a is an effective biomarker of CD8+ activation, degranulation and cytolytic function. Using isotype CAR-T cells, it was sought to be determined if the presence of the bispecific antibody targeting G₄S linker and PSMA could activate CAR-T cells in the presence of PSMA+ tumor cells. Isotype CAR-T cells were co-cultured with PSMA-expressing tumor cells in the presence or absence of BsAb (5 μg/ml). After 5-hours co-culture, increased CD107a expression was observed in the total cell population only in the presence of BsAb, suggesting that degranulation occurred in response to BsAb addition (FIG. 9C). CEN-63-13 antibody was used to detect G₄S-containing CAR-T cells (after washing). For both populations of CD8+ cells (CAR+CD8+ and CAR-CD8+), CD107a expression was compared. As shown in FIG. 9C, in the presence of BsAb, CAR+CD8+ cells were enriched for CD107a expression (as high as 21.2% of total), while far lower levels of CD107a were observed in absence of BsAb in both the CAR+ populations (without BsAb). Moreover, CD107a expression was undetectable in CD8+ CAR-cellular population, in the presence and absence of BsAb. These results demonstrated the increase of CD107a expression was mainly contributed by CD8+ CAR+ cells in the presence of BsAb.

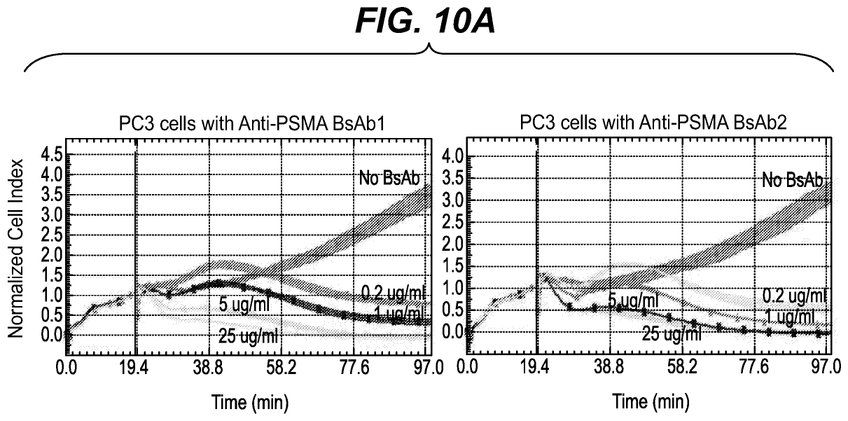

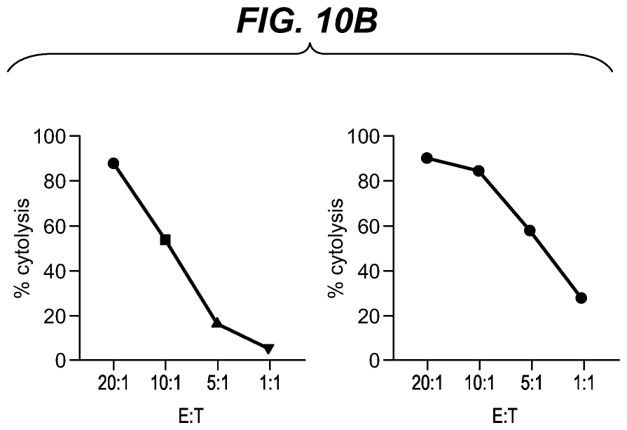

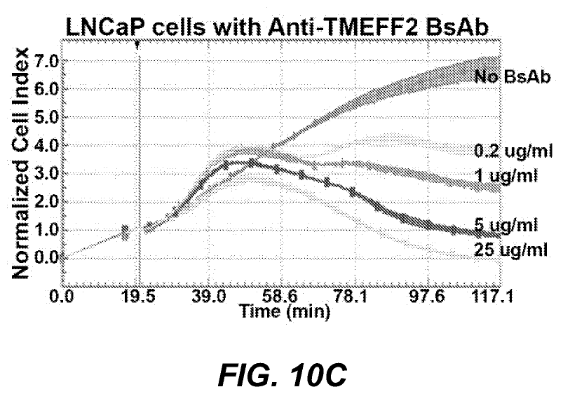

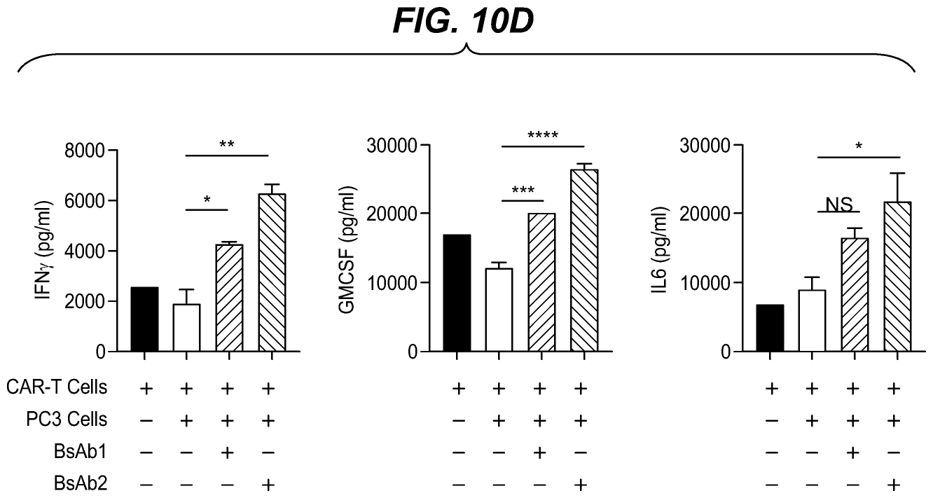

The ability of isotype ScFv bearing CAR-T cells to lyse tumor cells was next examined in the presence of BsAb. First, bispecific antibodies targeting PSMA and G₄S were utilized as conduit or adapter molecules. Anti-PSMA BsAb1 contains CEN-63-13 fab arms with an anti-PSMA ScFv appended to the heavy chain C-terminus. Anti-PSMA BsAb2 uses a reverse orientation, with anti-PSMA Fab arms and a C-terminal CEN-63-13 ScFv. When these anti-PSMA×anti G₄S BsAbs were titrated in the presence of isotype CAR-T cells and PSMA+PC3 cells (E:T ratio=5:1), tumor cell lysis was observed in a 97 hour time course using xCELLigence monitoring (FIG. 10A). In wells lacking BsAb, tumor growth continued unabated. Isotype CAR-T cells mediated tumor specific lysis in the presence of either BsAb.

In a separate experiment, the E:T ratio of CAR-T:PC3 cells was varied with a fixed BsAb concentration (5 μg/ml). These results show that isotype CAR-T BsAb specific killing varies with E:T ratio similarly to traditional CAR-T assays (FIG. 10B). Similar results were observed using an anti-TMEFF2 bispecific antibody containing anti-TMEFF2 fab arms with the CEN-63-13 ScFv appended to the heavy chain C-terminus. In this case, Isotype CAR-T cells were shown to kill LNCaP prostate derived tumor cells in the presence of BsAb (FIG. 10C).

Having demonstrated the cytotoxic potential of conduit CAR-T cells in the presence of bispecific antibodies, it was next sought to quantify whether cytokines commonly observed upon CAR-T activation were also being produced in the presence of BsAbs. IFNγ, IL-6, and GM-CSF levels produced by CAR-T cells were quantified by Intellicyt iQue measurement. CAR-T cells were co-cultured with tumor cells at E:T ratio of 5:1 and bispecific molecules were added at a final concentration of 5 μM. The addition of bispecific molecules significantly increased cytokine production by CAR-T cells in the presence of target cells (FIG. 10D). Interferon γ (IFNγ) levels were observed to approximately double in the presence of BsAb1 and triple in the presence of BsAb2. Granulocyte colony stimulating factor (GM-CSF) levels increased similarly (compared to CAR-T and PC3 alone). Interleukin-6 (IL-6) levels were significantly increased in the presence of BsAb2, but not BsAb1.

These increases in cytokine expression over controls suggest that conduit bispecifics were capable of directing T cells specifically to tumor cells resulting in T cell activation. CDRs for the inert scFv, H3-23/L1-39 are provided in Table 4.

TABLE 4
CDR sequences

| Ab | Chain | CDR1 (SEQ ID NO) | CDR2 (SEQ ID NO) | CDR3 (SEQ ID NO) |
|---|---|---|---|---|
| CEN-63-13 | Heavy | GFSLSSN (1) | GRSGS (2) | HFYL (3) |
| | Light | QASQSVYSNYLS (4) | TTSTLEP (5) | AGGYSVDIWV (6) |
| H3-23/L1-39 | Heavy | GFTFSSY (11) | SGSGGS (12) | AKYDGIYGELDF (13) |
| | Light | RASQSISSYLN (14) | AASSLQS (15) | QQSYSTPLT (16) |
| PS3B35 | Heavy | GYTFILY (19) | NPNNGG (20) | AAGWNLDY (21) |
| | Light | KASQDVGTAVD (22) | WASTRHT (23) | QQYNSYPLT (24) |
| TMEB570 | Heavy | GGTFSSYYIS (92) | GIIPISGRAN (93) | DGYSSGRSTTYAFDY (94) |
| | Light | RASQSVSTYYLA (95) | GASYRAT (96) | QQYGHSPIT (97) |

The tumor specific cytotoxicity of the conduit bispecific approach was demonstrated using an impedance-based cell viability assay. Tumor cells were adhered to electroconductive plates and impedance was measured over time. When no T cells were added, tumor cell mass increased exponentially. In the presence of both T cells and conduit bispecific antibodies, potent T cell mediated cytotoxicity at various Effector T cell to target ratios was observed.

These results taken together suggest that the conduit bispecific approach can direct CAR-T cells to tumor cells expressing tumor specific antigens. These CAR-T cells show increases in canonical cell surface activation markers and cytokine expression.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

It is claimed:

1. An isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequences of:
a. SEQ ID NOs:1, 2, 3, 4, 5, and 6, respectively;
wherein the monoclonal antibody or antigen-binding fragment thereof specifically binds a (G₄S)ₙ polypeptide linker, wherein n is at least 2.

2. The isolated monoclonal antibody or antigen-binding fragment thereof of claim 1, comprising a heavy chain variable region having a polypeptide sequence at least 95% identical to SEQ ID NO:7, or a light chain variable region having a polypeptide sequence at least 95% identical to SEQ ID NO:8.

3. The isolated monoclonal antibody or antigen-binding fragment thereof of claim 1 or 2, comprising:
a. a heavy chain variable region having the polypeptide sequence of SEQ ID NO:7, and a light chain variable region having the polypeptide sequence of SEQ ID NO:8.

4. The isolated monoclonal antibody or antigen-binding fragment thereof of any one of claims 1 to 3, wherein the antibody or antigen-binding fragment thereof is chimeric and/or human or humanized.

5. The isolated monoclonal antibody or antigen-binding fragment thereof of any one of claims 1 to 4, wherein the monoclonal antibody or antigen-binding fragment thereof is a single chain variable fragment (scFv).

6. The isolated monoclonal antibody or antigen-binding fragment thereof of claim 5, wherein the scFv comprises the amino acid sequence selected from SEQ ID NO:29 or SEQ ID NO:30.

7. An isolated nucleic acid encoding the monoclonal antibody or antigen-binding fragment thereof of any one of claims 1 to 6.

8. An isolated vector comprising the isolated nucleic acid of claim 7.

9. An isolated host cell comprising the vector of claim 8.

10. An isolated bispecific antibody or antigen-binding fragment thereof comprising a first polypeptide component and a second polypeptide component, wherein
a. the first polypeptide component comprises (i) a first antigen-binding domain that specifically binds a (G₄S)ₙ polypeptide linker, wherein n is at least 2, or (ii) a non-antigen binding single chain variable fragment (scFv) and a (G₄S)ₙ polypeptide linker, wherein n is at least 2; and
b. the second polypeptide component comprises a second antigen-binding domain that specifically binds a tumor associated antigen (TAA), preferably a human TAA

11. The isolated bispecific antibody or antigen-binding fragment thereof of claim 10, wherein
a. the first antigen-binding domain comprises a heavy chain complementarity determining region 1 (HCDR1), a HCDR2, a HCDR3, a light chain complementarity determining region 1 (LCDR1), a LCDR2, and a LCDR3 having the polypeptide sequences of SEQ ID NOs:1, 2, 3, 4, 5, and 6, respectively; and
b. the second antigen-binding domain comprises a heavy chain complementarity determining region 1 (HCDR1), a HCDR2, a HCDR3, a light chain complementarity determining region 1 (LCDR1), a LCDR2, and a LCDR3.

12. The isolated bispecific antibody or antigen-binding fragment thereof of claim 10 or 11, wherein the second antigen-binding domain specifically binds prostate-specific membrane antigen (PSMA), preferably human PSMA, or transmembrane protein with EGF-like and two follistatin-like domains 2 (TMEFF2), preferably human TMEFF2.

13. The isolated bispecific antibody or antigen-binding fragment thereof of any one of claims 10 to 12, wherein the second antigen-binding domain comprises a heavy chain complementarity determining region 1 (HCDR1), a HCDR2, a HCDR3, a light chain complementarity determining region having the polypeptide sequences of:
a. SEQ ID NOs:19, 20, 21, 22, 23, and 24, respectively; or
b. SEQ ID NOs:92, 93, 94, 95, 96, and 97, respectively.

14. The isolated bispecific antibody or antigen-binding fragment thereof of any one of claims 10 to 13, wherein:
a. the first antigen-binding domain comprises a first heavy chain variable region having a polypeptide sequence at least 95% identical to SEQ ID NO:7, and a first light chain variable region having a polypeptide sequence at least 95% identical to SEQ ID NO:8; and
b. the second antigen-binding domain comprises a second heavy chain variable region having a polypeptide sequence at least 95% identical to SEQ ID NO:25 or SEQ ID NO:90, and a second light chain variable region having a polypeptide sequence at least 95% identical to SEQ ID NO:26 or SEQ ID NO:91.

15. The isolated bispecific antibody or antigen-binding fragment thereof of any one of claims 10 to 14, wherein:
a. the first heavy chain variable region comprises the polypeptide sequence of SEQ ID NO:7, and the first light chain variable region comprises the polypeptide sequence of SEQ ID NO:8; and
b. the second heavy chain variable region comprises the polypeptide sequence of SEQ ID NO:25 or SEQ ID NO:90, and a second light chain variable region comprises the polypeptide sequence of SEQ ID NO:26 or SEQ ID NO:91.

16. The isolated bispecific antibody or antigen-binding fragment thereof of any one of claims 10 to 15, wherein the antibody or antigen-binding fragment thereof is chimeric and/or human or humanized.

17. The isolated bispecific antibody or antigen-binding fragment thereof of any one of claims 10 to 16, wherein the bispecific antibody or antigen-binding fragment thereof comprises the amino acid sequences selected from SEQ ID NO:35 and SEQ ID NO:28, SEQ ID NO:36 and SEQ ID NO:28, SEQ ID NO:37 and SEQ ID NO:27, SEQ ID NO:38 and SEQ ID NO:27, SEQ ID NO: 101 and SEQ ID NO: 28, SEQ ID NO: 102 and SEQ ID NO: 28, SEQ ID NO: 103 and SEQ ID NO: 98, or SEQ ID NO: 104 and SEQ ID NO: 98.

18. The isolated bispecific antibody or antigen-binding fragment thereof of claim 10, wherein the non-antigen binding scFv comprises a heavy chain complementarity determining region 1 (HCDR1), a HCDR2, a HCDR3, a light chain complementarity determining region 1, a LCDR2, and a LCDR3 having the polypeptide sequences of SEQ ID NOs:11, 12, 13, 14, 15, and 16, respectively.

19. The isolated bispecific antibody or antigen-binding fragment thereof of claim 18, wherein the non-antigen binding scFv comprises a heavy chain variable region having an amino acid sequence at least 95% identical to SEQ ID NO:17, and a light chain variable region having an amino acid sequence at least 95% identical to SEQ ID NO:18.

20. The isolated bispecific antibody or antigen-binding fragment thereof of claim 18 or 19, wherein the non-antigen binding scFv comprises a heavy chain variable region having the amino acid sequence of SEQ ID NO:17, and a light chain variable region having the amino acid sequence of SEQ ID NO:18.

21. The isolated bispecific antibody or antigen-binding fragment thereof of any one of claim 10 or 18 to 20, wherein the (G₄S)ₙ linker peptide comprises the amino acid sequence of SEQ ID NO:45.

22. An isolated nucleic acid sequence encoding the isolated bispecific antibody or antigen-binding fragment thereof of any one of claims 10 to 21.

23. An isolated vector comprising the isolated nucleic acid sequence of claim 22.

24. An isolated host cell comprising the isolated vector of claim 23.

25. An isolated polynucleotide comprising a nucleic acid encoding a chimeric antigen receptor (CAR), wherein the CAR comprises:
a. an extracellular domain comprising (1) a non-antigen binding single chain variable fragment (scFv) and a (G₄S)ₙ polypeptide linker or (2) an antigen binding domain that specifically binds a (G₄S)ₙ polypeptide linker;
b. a transmembrane region; and
c. an intracellular signaling domain.

26. The isolated polynucleotide of claim 25, wherein the non-antigen binding scFv comprises a heavy chain complementarity determining region 1 (HCDR1), a HCDR2, a HCDR3, a light chain complementarity determining region 1, a LCDR2, and a LCDR3 having the polypeptide sequences of SEQ ID NOs:11, 12, 13, 14, 15, and 16, respectively.

27. The isolated polynucleotide of claim 25 or 26, wherein the non-antigen binding scFv comprises a heavy chain variable region having an amino acid sequence at least 95% identical to SEQ ID NO:17, and a light chain variable region having an amino acid sequence at least 95% identical to SEQ ID NO:18.

28. The isolated polynucleotide of any one of claims 25 to 27, wherein the non-antigen binding scFv comprises a heavy chain variable region having the amino acid sequence of SEQ ID NO:17, and a light chain variable region having the amino acid sequence of SEQ ID NO:18.

29. The isolated polynucleotide of any one of claims 25 to 28, wherein the (G₄S)ₙ linker peptide comprises the amino acid sequence of SEQ ID NO:45.

30. The isolated polynucleotide of any one of claims 25 to 29, wherein the CAR comprises an amino acid sequence selected from SEQ ID NO:39 or SEQ ID NO:40.

31. The isolated polynucleotide of claim 25, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
a. SEQ ID NOs:1, 2, 3, 4, 5, and 6, respectively;
wherein the antigen binding domain specifically binds a (G₄S)ₙ polypeptide linker, wherein n is at least 2

32. The isolated polynucleotide of claim 31, wherein the antigen binding domain comprises a heavy chain variable region having a polypeptide sequence at least 95% identical to SEQ ID NO:7, or a light chain variable region having a polypeptide sequence at least 95% identical to SEQ ID NO:8.

33. The isolated polynucleotide of claim 31 or 32, wherein the antigen binding domain comprises:
a. a heavy chain variable region having the polypeptide sequence of SEQ ID NO:7, and a light chain variable region having the polypeptide sequence of SEQ ID NO:8.

34. The isolated polynucleotide of any one of claims 31 to 33, wherein the antigen binding domain is chimeric and/or human or humanized.

35. The isolated polynucleotide of any one of claims 31 to 34, wherein the antigen binding domain is a single chain variable fragment (scFv).

36. The isolated polynucleotide of claim 35, wherein the scFv comprises the amino acid sequence selected from SEQ ID NO:29 or SEQ ID NO:30

37. A chimeric antigen receptor (CAR) encoded by the isolated polynucleotide of any one of claims 25 to 36.

38. An isolated vector comprising the isolated polynucleotide of any one of claims 25 to 36.

39. An isolated host cell comprising the isolated vector of claim 38.

40. The host cell of claim 39, wherein the host cell is a T cell, preferably a human T cell.

41. A kit comprising:
a. an isolated polynucleotide comprising a nucleic acid encoding a chimeric antigen receptor (CAR), wherein the CAR comprises:
i. an extracellular domain comprising (1) a non-antigen binding single chain variable fragment (scFv) and a (G₄S)ₙ polypeptide linker or (2) an antigen binding domain that specifically binds a (G₄S)ₙ polypeptide linker;
ii. a transmembrane region; and
iii. an intracellular signaling domain; and
b. the isolated bispecific antibody or antigen-binding fragment thereof of any one of claims 10 to 21.

42. The kit of claim 41, wherein the non-antigen binding scFv comprises a heavy chain complementarity determining region 1 (HCDR1), a HCDR2, a HCDR3, a light chain complementarity determining region 1, a LCDR2, and a LCDR3 having the polypeptide sequences of SEQ ID NOs:11, 12, 13, 14, 15, and 16, respectively

43. The kit of claim 42, wherein the non-antigen binding scFv comprises a heavy chain variable region having the amino acid sequence of SEQ ID NO:17, and a light chain variable region having the amino acid sequence of SEQ ID NO:18.

44. The kit of any one of claims 41 to 43, wherein the (G₄S)ₙ linker peptide comprises the amino acid sequence of SEQ ID NO:45.

45. The kit of any one of claims 41 to 44, wherein the CAR comprises an amino acid sequence selected from SEQ ID NO:39 or SEQ ID NO:40.

46. A method of treating a cancer expressing a tumor associated antigen (TAA) in a subject in need thereof, the method comprising administering to the subject the isolated host cell of claim 39 and a pharmaceutical composition comprising a bispecific antibody or antigen-binding fragment thereof and a pharmaceutically acceptable carrier,
wherein the bispecific antibody or antigen binding fragment thereof comprises a first polypeptide component and a second polypeptide component, wherein
a. the first polypeptide component comprises (i) a first antigen-binding domain that specifically binds a (G₄S)ₙ polypeptide linker, wherein n is at least 2, or (ii) a non-antigen binding single chain variable fragment (scFv) and a (G₄S)ₙ polypeptide linker, wherein n is at least 2; and
b. the second polypeptide component comprises a second antigen-binding domain that specifically binds a tumor associated antigen (TAA), preferably a human TAA.

47. The method of claim 46, wherein the bispecific antibody or antigen-binding fragment thereof, wherein:
a. the first antigen-binding domain comprises a heavy chain complementarity determining region 1 (HCDR1), a HCDR2, a HCDR3, a light chain complementarity determining region 1 (LCDR1), a LCDR2, and a LCDR3 having the polypeptide sequences of SEQ ID NOs:1, 2, 3, 4, 5, and 6, respectively; and
b. the second antigen-binding domain comprises a heavy chain complementarity determining region 1 (HCDR1), a HCDR2, a HCDR3, a light chain complementarity determining region 1 (LCDR1), a LCDR2, and a LCDR3.

48. The method of claim 46 or 47, wherein the second antigen-binding domain specifically binds prostate-specific membrane antigen (PSMA), preferably human PSMA, or transmembrane protein with EGF-like and two follistatin-like domains 2 (TMEFF2), preferably human TMEFF2.

49. The method of any one of claims 46 to 48, wherein the second antigen-binding domain comprises a heavy chain complementarity determining region 1 (HCDR1), a HCDR2, a HCDR3, a light chain complementarity determining region having the polypeptide sequences of:
a. SEQ ID NOs:19, 20, 21, 22, 23, and 24, respectively; or
b. SEQ ID NOs:92, 93, 94, 95, 96, and 97, respectively.

50. The method of any one of claims 46 to 49, wherein the bispecific antibody or antigen binding fragment thereof comprises:
a. a first heavy chain variable region having the polypeptide sequence of SEQ ID NO:7, and a first light chain variable region having the polypeptide sequence of SEQ ID NO:8; and
b. a second heavy chain variable region having the polypeptide sequence of SEQ ID NO:25 or SEQ ID NO:90, and a second light chain variable region having the polypeptide sequence of SEQ ID NO:26 or SEQ ID NO:91.


SEQUENCE LISTING (104 sequences)

SEQ ID NO: 1 - protein, 7 aa, Artificial Sequence
  CEN-63-13 HCDR1
GFSLSSN

SEQ ID NO: 2 - protein, 5 aa, Artificial Sequence
  CEN-63-13 HCDR2
GRSGS

SEQ ID NO: 3 - protein, 4 aa, Artificial Sequence
  CEN-63-13 HCDR3
HFYL

SEQ ID NO: 4 - protein, 12 aa, Artificial Sequence
  CEN-63-13 LCDR1
QASQSVYSNYLS

SEQ ID NO: 5 - protein, 7 aa, Artificial Sequence
  CEN-63-13 LCDR2
TTSTLEP

SEQ ID NO: 6 - protein, 10 aa, Artificial Sequence
  CEN-63-13 LCDR3
AGGYSVDIWV

SEQ ID NO: 7 - protein, 110 aa, Artificial Sequence
  CEN-63-13 Variable heavy chain region (VH)
QSVKESGGRLVTPGTPLTLTCTVSGFSLSSNAIDWVRQAPGKGLEWIGILGRSGSTYYASWAKGRFTISRTSSTTVDLKITSPTTEDTATYFCARHFYLWGPGTLVTVSS

SEQ ID NO: 8 - protein, 109 aa, Artificial Sequence
  CEN-63-13 Variable light (VL) chain region (kappa)
AQVLTQTASPVSAAVGGTVTINCQASQSVYSNYLSWYQQKPGQPPKLLMATTSTLEPGVPSRFKGSGSGTQFTLTISDLECDDAATYYCAGGYSVDIWVFGGGTEVVVK

SEQ ID NO: 9 - protein, 459 aa, Artificial Sequence
  CEN-63-13 HC
MAWVWTLLFLMAAAQSIQAQSVKESGGRLVTPGTPLTLTCTVSGFSLSSNAIDWVRQAPGKGLEWIGILGRSGSTYYASWAKGRFTISRTSSTTVDLKITSPTTEDTATYFCARHFYLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 235 aa, Artificial Sequence
  CEN-63-13 LC
MAWVWTLLFLMAAAQSIQAAQVLTQTASPVSAAVGGTVTINCQASQSVYSNYLSWYQQKPGQPPKLLMATTSTLEPGVPSRFKGSGSGTQFTLTISDLECDDAATYYCAGGYSVDIWVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 7 aa, Artificial Sequence
  H3-23/L1-39 HCDR1
GFTFSSY

SEQ ID NO: 12 - protein, 6 aa, Artificial Sequence
  H3-23/L1-39 HCDR2
SGSGGS

SEQ ID NO: 13 - protein, 12 aa, Artificial Sequence
  H3-23/L1-39 HCDR3
AKYDGIYGELDF

SEQ ID NO: 14 - protein, 11 aa, Artificial Sequence
  H3-23/L1-39 LCDR1
RASQSISSYLN

SEQ ID NO: 15 - protein, 7 aa, Artificial Sequence
  H3-23/L1-39 LCDR2
AASSLQS

SEQ ID NO: 16 - protein, 9 aa, Artificial Sequence
  H3-23/L1-39 LCDR3
QQSYSTPLT

SEQ ID NO: 17 - protein, 119 aa, Artificial Sequence
  H3-23/L1-39 Variable heavy chain region (VH)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGQGTLVTVSS

SEQ ID NO: 18 - protein, 107 aa, Artificial Sequence [sequence omitted]

SEQ ID NO: 19 - protein, 7 aa, Artificial Sequence
  PS3B35 HCDR1
GYTFTEY

SEQ ID NO: 20 - protein, 6 aa, Artificial Sequence
  PS3B35 HCDR2
NPNNGG

SEQ ID NO: 21 - protein, 8 aa, Artificial Sequence
  PS3B35 HCDR3
AAGWNFDY

SEQ ID NO: 22 - protein, 11 aa, Artificial Sequence
  PS3B35 LCDR1
KASQDVGTAVD

SEQ ID NO: 23 - protein, 7 aa, Artificial Sequence
  PS3B35 LCDR2
WASTRHT

SEQ ID NO: 24 - protein, 9 aa, Artificial Sequence
  PS3B35 LCDR3
QQYNSYPLT

SEQ ID NO: 25 - protein, 115 aa, Artificial Sequence
  PS3B35 Variable heavy chain region (VH)
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 26 - protein, 107 aa, Artificial Sequence
  PS3B35 Variable light (VL) chain region (kappa)
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIK

SEQ ID NO: 27 - protein, 233 aa, Artificial Sequence
  PS3B35 VK (variable light chain region kappa)
MAWVWTLLFLMAAAQSIQADIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 235 aa, Artificial Sequence; identical to SEQ ID NO: 10
  CEN-63-13 VK (variable light chain region kappa)

SEQ ID NO: 29 - protein, 235 aa, Artificial Sequence
  CEN-63-13 scFv VH-VL
QSVKESGGRLVTPGTPLTLTCTVSGFSLSSNAIDWVRQAPGKGLEWIGILGRSGSTYYASWAKGRFTISRTSSTTVDLKITSPTTEDTATYFCARHFYLWGPGTLVTVSSGTEGKSSGSGSESKSTAQVLTQTASPVSAAVGGTVTINCQASQSVYSNYLSWYQQKPGQPPKLLMATTSTLEPGVPSRFKGSGSGTQFTLTISDLECDDAATYYCAGGYSVDIWVFGGGTEVVVK

SEQ ID NO: 30 - protein, 235 aa, Artificial Sequence
  CEN-63-13 scFv VL-VH
AQVLTQTASPVSAAVGGTVTINCQASQSVYSNYLSWYQQKPGQPPKLLMATTSTLEPGVPSRFKGSGSGTQFTLTISDLECDDAATYYCAGGYSVDIWVFGGGTEVVVKGTEGKSSGSGSESKSTQSVKESGGRLVTPGTPLTLTCTVSGFSLSSNAIDWVRQAPGKGLEWIGILGRSGSTYYASWAKGRFTISRTSSTTVDLKITSPTTEDTATYFCARHFYLWGPGTLVTVSS

SEQ ID NO: 31 - protein, 238 aa, Artificial Sequence
  PS3B35 scFv VH-VL
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSGTEGKSSGSGSESKSTDIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIK

SEQ ID NO: 32 - protein, 238 aa, Artificial Sequence
  PS3B35 scFv VL-VH
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKGTEGKSSGSGSESKSTEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 33 - protein, 246 aa, Artificial Sequence
  H3-23/L1-39 scFv VH-VL
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 34 - protein, 246 aa, Artificial Sequence
  H3-23/L1-39 scFv VL-VH
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGQGTLVTVSS

SEQ ID NO: 35 - protein, 705 aa, Artificial Sequence
  MSCB336
MAWVWTLLFLMAAAQSIQAQSVKESGGRLVTPGTPLTLTCTVSGFSLSSNAIDWVRQAPGKGLEWIGILGRSGSTYYASWAKGRFTISRTSSTTVDLKITSPTTEDTATYFCARHFYLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGAGGAGGAGDIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKGTEGKSSGSGSESKSTEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 36 - protein, 705 aa, Artificial Sequence
  PTCB330
MAWVWTLLFLMAAAQSIQAQSVKESGGRLVTPGTPLTLTCTVSGFSLSSNAIDWVRQAPGKGLEWIGILGRSGSTYYASWAKGRFTISRTSSTTVDLKITSPTTEDTATYFCARHFYLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGAGGAGGAGEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSGTEGKSSGSGSESKSTDIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIK

SEQ ID NO: 37 - protein, 707 aa, Artificial Sequence
  MSCB337
MAWVWTLLFLMAAAQSIQAEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGAGGAGGAGQSVKESGGRLVTPGTPLTLTCTVSGFSLSSNAIDWVRQAPGKGLEWIGILGRSGSTYYASWAKGRFTISRTSSTTVDLKITSPTTEDTATYFCARHFYLWGPGTLVTVSSGTEGKSSGSGSESKSTAQVLTQTASPVSAAVGGTVTINCQASQSVYSNYLSWYQQKPGQPPKLLMATTSTLEPGVPSRFKGSGSGTQFTLTISDLECDDAATYYCAGGYSVDIWVFGGGTEVVVK

SEQ ID NO: 38 - protein, 707 aa, Artificial Sequence
  MSCB338
MAWVWTLLFLMAAAQSIQAEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGAGGAGGAGAQVLTQTASPVSAAVGGTVTINCQASQSVYSNYLSWYQQKPGQPPKLLMATTSTLEPGVPSRFKGSGSGTQFTLTISDLECDDAATYYCAGGYSVDIWVFGGGTEVVVKGTEGKSSGSGSESKSTQSVKESGGRLVTPGTPLTLTCTVSGFSLSSNAIDWVRQAPGKGLEWIGILGRSGSTYYASWAKGRFTISRTSSTTVDLKITSPTTEDTATYFCARHFYLWGPGTLVTVSS

SEQ ID NO: 39 - protein, 488 aa, Artificial Sequence
  H3-23/L1-39 scFv LH with G4S linker CAR-T
MAWVWTLLFLMAAAQSIQADIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGQGTLVTVSSTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 488 aa, Artificial Sequence
  H3-23/L1-39 scFv HL with G4S linker CAR-T
MAWVWTLLFLMAAAQSIQAEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTSTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 45 aa, Artificial Sequence
  CD8 extracellular domain
TSTPAPRPPTPAPTIASQPLSLRPEACRPAAGGAVHTRGLDFACD

SEQ ID NO: 42 - protein, 34 aa, Artificial Sequence
  CD8 transmembrane domain
IYIWAPLAGTCGVLLLSLVITLYCKRGRKKLLYI

SEQ ID NO: 43 - protein, 31 aa, Artificial Sequence
  CD137 costimulatory domain
FKQPFMRPVQTTQEEDGCSCRFPEEEEGGCE

SEQ ID NO: 44 - protein, 113 aa, Artificial Sequence
  CD3 zeta
LRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 20 aa, Artificial Sequence
  G4S peptide linker (20AA)
GGGGSGGGGSGGGGSGGGGS

SEQ ID NO: 46 - protein, 19 aa, Artificial Sequence
  G4S peptide linker (19AA)
GGGGSGGGGSGGGGSGGGG

SEQ ID NO: 47 - protein, 18 aa, Artificial Sequence
  G4S peptide linker (18AA)
GGGGSGGGGSGGGGSGGG

SEQ ID NO: 48 - protein, 17 aa, Artificial Sequence
  G4S peptide linker (17AA)
GGGGSGGGGSGGGGSGG

SEQ ID NO: 49 - protein, 16 aa, Artificial Sequence
  G4S peptide linker (16AA)
GGGGSGGGGSGGGGSG

SEQ ID NO: 50 - protein, 15 aa, Artificial Sequence
  G4S peptide linker (15AA)
GGGGSGGGGSGGGGS

SEQ ID NO: 51 - protein, 14 aa, Artificial Sequence
  G4S peptide linker (14AA)
GGGGSGGGGSGGGG

SEQ ID NO: 52 - protein, 13 aa, Artificial Sequence
  G4S peptide linker (13AA)
GGGGSGGGGSGGG

SEQ ID NO: 53 - protein, 12 aa, Artificial Sequence
  G4S peptide linker (12AA)
GGGGSGGGGSGG

SEQ ID NO: 54 - protein, 11 aa, Artificial Sequence
  G4S peptide linker (11AA)
GGGGSGGGGSG

SEQ ID NO: 55 - protein, 10 aa, Artificial Sequence
  G4S peptide linker (10AA)
GGGGSGGGGS

SEQ ID NO: 56 - protein, 9 aa, Artificial Sequence
  GAG peptide linker
GAGGAGGAG

SEQ ID NO: 57 - protein, 16 aa, Artificial Sequence
  scFv linker
GTEGKSSGSGSESKST

SEQ ID NO: 58 - protein, 20 aa, Artificial Sequence
  L1 peptide linker
GGSEGKSSGSGSESKSTGGS

SEQ ID NO: 59 - protein, 8 aa, Artificial Sequence
  L2 peptide linker
GGGSGGGS

SEQ ID NO: 60 - protein, 12 aa, Artificial Sequence
  L3 peptide linker
GGGSGGGSGGGS

SEQ ID NO: 61 - protein, 16 aa, Artificial Sequence
  L4 peptide linker
GGGSGGGSGGGSGGGS

SEQ ID NO: 62 - protein, 20 aa, Artificial Sequence
  L5 peptide linker
GGGSGGGSGGGSGGGSGGGS

SEQ ID NO: 63 - protein, 25 aa, Artificial Sequence
  L8 peptide linker
GGGGSGGGGSGGGGSGGGGSGGGGS

SEQ ID NO: 64 - protein, 18 aa, Artificial Sequence
  L9 peptide linker
GSTSGSGKPGSGEGSTKG

SEQ ID NO: 65 - protein, 14 aa, Artificial Sequence
  L10 peptide linker
IRPRAIGGSKPRVA

SEQ ID NO: 66 - protein, 15 aa, Artificial Sequence
  L11 peptide linker
GKGGSGKGGSGKGGS

SEQ ID NO: 67 - protein, 15 aa, Artificial Sequence
  L12 peptide linker
GGKGSGGKGSGGKGS

SEQ ID NO: 68 - protein, 15 aa, Artificial Sequence
  L13 peptide linker
GGGKSGGGKSGGGKS

SEQ ID NO: 69 - protein, 15 aa, Artificial Sequence
  L14 peptide linker
GKGKSGKGKSGKGKS

SEQ ID NO: 70 - protein, 15 aa, Artificial Sequence
  L15 peptide linker
GGGKSGGKGSGKGGS

SEQ ID NO: 71 - protein, 15 aa, Artificial Sequence
  L16 peptide linker
GKPGSGKPGSGKPGS

SEQ ID NO: 72 - protein, 20 aa, Artificial Sequence
  L17 peptide linker
GKPGSGKPGSGKPGSGKPGS

SEQ ID NO: 73 - protein, 20 aa, Artificial Sequence
  L18 peptide linker
GKGKSGKGKSGKGKSGKGKS

SEQ ID NO: 74 - protein, 14 aa, Artificial Sequence
  L19 peptide linker
STAGDTHLGGEDFD

SEQ ID NO: 75 - protein, 15 aa, Artificial Sequence
  L20 peptide linker
GEGGSGEGGSGEGGS

SEQ ID NO: 76 - protein, 15 aa, Artificial Sequence
  L21 peptide linker
GGEGSGGEGSGGEGS

SEQ ID NO: 77 - protein, 15 aa, Artificial Sequence
  L22 peptide linker
GEGESGEGESGEGES

SEQ ID NO: 78 - protein, 15 aa, Artificial Sequence
  L23 peptide linker
GGGESGGEGSGEGGS

SEQ ID NO: 79 - protein, 20 aa, Artificial Sequence
  L24 peptide linker
GEGESGEGESGEGESGEGES

SEQ ID NO: 80 - protein, 18 aa, Artificial Sequence
  L25 peptide linker
GSTSGSGKPGSGEGSTKG

SEQ ID NO: 81 - protein, 19 aa, Artificial Sequence
  L26 peptide linker
PRGASKSGSASQTGSAPGS

SEQ ID NO: 82 - protein, 19 aa, Artificial Sequence
  L27 peptide linker
GTAAAGAGAAGGAAAGAAG

SEQ ID NO: 83 - protein, 19 aa, Artificial Sequence
  L28 peptide linker
GTSGSSGSGSGGSGSGGGG

SEQ ID NO: 84 - protein, 20 aa, Artificial Sequence
  L29 peptide linker
GKPGSGKPGSGKPGSGKPGS

SEQ ID NO: 85 - protein, 4 aa, Artificial Sequence
  L30 peptide linker
GSGS

SEQ ID NO: 86 - protein, 10 aa, Artificial Sequence
  L31 peptide linker
APAPAPAPAP

SEQ ID NO: 87 - protein, 20 aa, Artificial Sequence
  L32 peptide linker
APAPAPAPAPAPAPAPAPAP

SEQ ID NO: 88 - protein, 32 aa, Artificial Sequence
  L33 peptide linker
AEAAAKEAAAKEAAAAKEAAAAKEAAAAKAAA

SEQ ID NO: 89 - protein, 5 aa, Artificial Sequence
  GGGGS motif
GGGGS

SEQ ID NO: 90 - protein, 124 aa, Artificial Sequence
  TMEB570 Variable Heavy Chain Region (VH)
QVQLVQSGAEVKKPGSSVKVSCKASGGTFSSYYISWVRQAPGQGLEWMGGIIPISGRANYAQKFQGRVTITADESTSTAYMELSSLRSEDTAVYYCARDGYSSGRSTTYAFDYWGQGTLVTVSS

SEQ ID NO: 91 - protein, 108 aa, Artificial Sequence
  TMEB570 Variable Light (VL) Chain Region kappa
EIVLTQSPGTLSLSPGERATLSCRASQSVSTYYLAWYQQKPGQAPRLLIYGASYRATGIPDRFSGSGSGTDFTLTISRLEPEDFAVYYCQQYGHSPITFGQGTKVEIK

SEQ ID NO: 92 - protein, 10 aa, Artificial Sequence
  TMEB570 HCDR1
GGTFSSYYIS

SEQ ID NO: 93 - protein, 10 aa, Artificial Sequence
  TMEB570 HCDR2
GIIPISGRAN

SEQ ID NO: 94 - protein, 15 aa, Artificial Sequence
  TMEB570 HCDR3
DGYSSGRSTTYAFDY

SEQ ID NO: 95 - protein, 12 aa, Artificial Sequence
  TMEB570 LCDR1
RASQSVSTYYLA

SEQ ID NO: 96 - protein, 7 aa, Artificial Sequence
  TMEB570 LCDR2
GASYRAT

SEQ ID NO: 97 - protein, 9 aa, Artificial Sequence
  TMEB570 LCDR3
QQYGHSPIT

SEQ ID NO: 98 - protein, 234 aa, Artificial Sequence
  TMEB570 VK (Variable Light Chain Region Kappa)
MAWVWTLLFLMAAAQSIQAEIVLTQSPGTLSLSPGERATLSCRASQSVSTYYLAWYQQKPGQAPRLLIYGASYRATGIPDRFSGSGSGTDFTLTISRLEPEDFAVYYCQQYGHSPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 248 aa, Artificial Sequence
  TMEB570 scFv VH-VL
QVQLVQSGAEVKKPGSSVKVSCKASGGTFSSYYISWVRQAPGQGLEWMGGIIPISGRANYAQKFQGRVTITADESTSTAYMELSSLRSEDTAVYYCARDGYSSGRSTTYAFDYWGQGTLVTVSSGTEGKSSGSGSESKSTEIVLTQSPGTLSLSPGERATLSCRASQSVSTYYLAWYQQKPGQAPRLLIYGASYRATGIPDRFSGSGSGTDFTLTISRLEPEDFAVYYCQQYGHSPITFGQGTKVEIK

SEQ ID NO: 100 - protein, 248 aa, Artificial Sequence
  TMEB570 scFv VL-VH
EIVLTQSPGTLSLSPGERATLSCRASQSVSTYYLAWYQQKPGQAPRLLIYGASYRATGIPDRFSGSGSGTDFTLTISRLEPEDFAVYYCQQYGHSPITFGQGTKVEIKGTEGKSSGSGSESKSTQVQLVQSGAEVKKPGSSVKVSCKASGGTFSSYYISWVRQAPGQGLEWMGGIIPISGRANYAQKFQGRVTITADESTSTAYMELSSLRSEDTAVYYCARDGYSSGRSTTYAFDYWGQGTLVTVSS

SEQ ID NO: 101 - protein, 715 aa, Artificial Sequence
  MSCB542
MAWVWTLLFLMAAAQSIQAQSVKESGGRLVTPGTPLTLTCTVSGFSLSSNAIDWVRQAPGKGLEWIGILGRSGSTYYASWAKGRFTISRTSSTTVDLKITSPTTEDTATYFCARHFYLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGAGGAGGAGEIVLTQSPGTLSLSPGERATLSCRASQSVSTYYLAWYQQKPGQAPRLLIYGASYRATGIPDRFSGSGSGTDFTLTISRLEPEDFAVYYCQQYGHSPITFGQGTKVEIKGTEGKSSGSGSESKSTQVQLVQSGAEVKKPGSSVKVSCKASGGTFSSYYISWVRQAPGQGLEWMGGIIPISGRANYAQKFQGRVTITADESTSTAYMELSSLRSEDTAVYYCARDGYSSGRSTTYAFDYWGQGTLVTVSS

SEQ ID NO: 102 - protein, 715 aa, Artificial Sequence
  MSCB543
MAWVWTLLFLMAAAQSIQAQSVKESGGRLVTPGTPLTLTCTVSGFSLSSNAIDWVRQAPGKGLEWIGILGRSGSTYYASWAKGRFTISRTSSTTVDLKITSPTTEDTATYFCARHFYLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGAGGAGGAGQVQLVQSGAEVKKPGSSVKVSCKASGGTFSSYYISWVRQAPGQGLEWMGGIIPISGRANYAQKFQGRVTITADESTSTAYMELSSLRSEDTAVYYCARDGYSSGRSTTYAFDYWGQGTLVTVSSGTEGKSSGSGSESKSTEIVLTQSPGTLSLSPGERATLSCRASQSVSTYYLAWYQQKPGQAPRLLIYGASYRATGIPDRFSGSGSGTDFTLTISRLEPEDFAVYYCQQYGHSPITFGQGTKVEIK

SEQ ID NO: 103 - protein, 716 aa, Artificial Sequence
  MSCB544
MAWVWTLLFLMAAAQSIQAQVQLVQSGAEVKKPGSSVKVSCKASGGTFSSYYISWVRQAPGQGLEWMGGIIPISGRANYAQKFQGRVTITADESTSTAYMELSSLRSEDTAVYYCARDGYSSGRSTTYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGAGGAGGAGQSVKESGGRLVTPGTPLTLTCTVSGFSLSSNAIDWVRQAPGKGLEWIGILGRSGSTYYASWAKGRFTISRTSSTTVDLKITSPTTEDTATYFCARHFYLWGPGTLVTVSSGTEGKSSGSGSESKSTAQVLTQTASPVSAAVGGTVTINCQASQSVYSNYLSWYQQKPGQPPKLLMATTSTLEPGVPSRFKGSGSGTQFTLTISDLECDDAATYYCAGGYSVDIWVFGGGTEVVVK

SEQ ID NO: 104 - protein, 716 aa, Artificial Sequence
  MSCB545
MAWVWTLLFLMAAAQSIQAQVQLVQSGAEVKKPGSSVKVSCKASGGTFSSYYISWVRQAPGQGLEWMGGIIPISGRANYAQKFQGRVTITADESTSTAYMELSSLRSEDTAVYYCARDGYSSGRSTTYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGAGGAGGAGAQVLTQTASPVSAAVGGTVTINCQASQSVYSNYLSWYQQKPGQPPKLLMATTSTLEPGVPSRFKGSGSGTQFTLTISDLECDDAATYYCAGGYSVDIWVFGGGTEVVVKGTEGKSSGSGSESKSTQSVKESGGRLVTPGTPLTLTCTVSGFSLSSNAIDWVRQAPGKGLEWIGILGRSGSTYYASWAKGRFTISRTSSTTVDLKITSPTTEDTATYFCARHFYLWGPGTLVTVSS